(19) United States
(12) Patent Application Publication
(10) Pub. No.: US 2022/0184124 A1
(43) Pub. Date: Jun. 16, 2022

(54) METHODS AND REAGENTS FOR CHARACTERIZING CAR T CELLS FOR THERAPIES

(71) Applicant: Allogene Therapeutics, Inc., South San Francisco, CA (US)

(72) Inventors: Lillian SEU, Daly City, CA (US); Melanie L. GEE, Cupertino, CA (US); Michael A. BOWEN, Seattle, WA (US)

(21) Appl. No.: 17/550,311

(22) Filed: Dec. 14, 2021

Related U.S. Application Data
(60) Provisional application No. 63/265,086, filed on Dec. 7, 2021.
(60) Provisional application No. 63/125,149, filed on Dec. 14, 2020.

(51) Int. Cl. A61K 35/17; C07K 14/725; C07K 14/705

(52) U.S. Cl. CPC A61K 35/17; C07K 14/70596; C07K 14/7051

(57) ABSTRACT

Provided herein are methods, kits and reagents for analyzing attributes of engineered immune cells, such as CAR T cells. For example, provided herein are methods of determining the amount or percentage of residual TCRαβ+ CAR T cells in allogeneic CAR T cell drug product and characterizing other important attributes of CAR T cell drug product.

### CROSS REFERENCE TO RELATED APPLICATIONS

The present application claims the benefit of priority to U.S. Provisional Application No. 63/125,149, filed on Dec. 14, 2020; and U.S. Provisional Application No. 63/265,086, filed on Dec. 7, 2021, the contents of both of which are hereby incorporated by reference in their entireties.

### SEQUENCE LISTING

This application contains a Sequence Listing which has been submitted electronically in ASCII format and is hereby incorporated by reference in its entirety. Said ASCII copy, created on Dec. 10, 2021, is named AT-037_03US_SL.txt and is 8,871 bytes in size.

### BACKGROUND

Chimeric antigen receptor (CAR) T cell therapy has achieved unprecedent success, yet manufacturing of CAR T cells also presents unprecedent challenges. CAR T cells derived from allogeneic donor cells (allogeneic CAR T cells) can be produced as off-the-shelf products with reduced costs and simplified manufacturing process as compared to CAR T cells derived from patient's own cells (autologous CAR T cells). Despite the advantages, unique challenges exist in allogeneic CAR T cells manufacturing. In the allogeneic setting, one way to mitigate graft v. host diseases (GvHD) is to disrupt TCRα, and/or TCRβ gene to reduce or eliminate functional TCRαβ signaling in the donor cells. The engineered CAR T cells are processed to remove or deplete any remaining unmodified TCRαβ positive cells, and any residual TCRαβ positive cells in the drug substance or final drug product is determined.

Hierarchical gating applied to semi-quantitative flow cytometry allows detailed analysis of heterogenous cell populations. The methodology is thus frequently used analytically to provide CMC (Chemistry, Manufacturing, and Controls) technical information of complex engineered cell therapeutics, such as CAR T cells, during or after manufacturing. But there are challenges when using these methods in a highly streamlined manufacturing setting in compliance with the cGMP (current Good Manufacture Practices) requirements. One source of the challenge is the variability of the starting cells. In the allogeneic CAR T cell manufacturing process, for example, the levels of TCRαβ positivity may vary in different donors to make gate-setting difficult. Inappropriate TCRαβ gate-setting may lead to under-reporting or over-reporting of TCRαβ positive T cells, potentially resulting in an increased risk of GvHD in patients, or an increased risk of rejecting suitable T cell drug product, respectively. Further, compatible reagents for TCR depletion and detection may not always be available.

In addition, it is desirable to determine other attributes of complex engineered cell therapeutics, such as CAR T drug products in ways amenable to the highly streamlined commercial manufacturing setting. Thus, there exists a need for improved methods for analyzing and characterizing important attributes of a complex cell therapeutic drug product in a manufacturing setting, especially in a GMP setting.

### TECHNICAL FIELD

The instant disclosure relates to methods and reagents for analyzing engineered immune cells, including those comprising chimeric antigen receptors (CARs), i.e., CAR T cells. For example, the instant disclosure relates to, inter alia, methods of determining the amount or percentage of residual TCRαβ+ CAR T cells in allogeneic CAR T cell drug product and methods of characterizing other attributes of CAR T cells.

### SUMMARY

The instant disclosure relates to methods and reagents for analyzing engineered immune cells, including those comprising chimeric antigen receptors (CARs).

Provided herein are methods, reagents and kits for analyzing and characterizing engineered immune cells, such as CAR T cells, in or after a manufacturing process. For example, provided herein are methods, reagents and kits for analyzing potency or polyfunctionality and/or other attributes of manufactured CAR T cells, including the amount or percentage of remaining TCRαβ+ T cells in manufactured allogeneic CAR T cells.

In one aspect, the present disclosure provides a method of analyzing a population of immune cells, wherein the population of immune cells has been engineered to introduce one or more genetic modifications at the TCRα, and/or TCRβ locus to reduce or impair TCRαβ surface expression, the method comprising the steps of (a) obtaining or measuring viable CD45+ cells from the population of immune cells; (b) obtaining or measuring CD5+/CD3+ cells from the cells in step (a); (c) measuring or determining a percentage or amount of CD3+/TCRγδ− cells from the cells in step (b), wherein the percentage or amount of CD3+/TCRγδ− cells in step (c) indicates a percentage or amount of TCRαβ+ T cells present in the population of immune cells. In some embodiments, the one or more genetic modifications are at the TCRα, locus. In some embodiments, the one or more genetic modifications are at the TRAC locus (TCRα, chain constant region).

In another aspect, the present disclosure provides a method of measuring a percentage or amount of TCRαβ+ T cells in a population of immune cells, wherein the population of immune cells has been engineered to introduce one or more genetic modifications at the TCRα, and/or TCRβ locus to reduce or impair TCRαβ surface expression, the method comprising the steps of (a) obtaining or measuring viable CD45+ cells from the population of immune cells; (b) obtaining or measuring CD5+/CD3+ cells from the cells in step (a); and (c) measuring or determining a percentage or amount of CD3+/TCRγδ− cells from the cells in step (b), wherein the percentage or amount of CD3+/TCRγδ− cells in step (c) indicates the percentage or amount of TCRαβ+ T cells in the population of immune cells. In some embodiments, the one or more genetic modifications are at the TCRα, locus. In some embodiments, the one or more genetic modifications are at the TRAC locus.

In some embodiments, the population of immune cells has been engineered to express a CAR. In some embodiments, the population of immune cells is CAR T cells. In some embodiments, the population of immune cells is peripheral blood mononuclear cells (PBMC). In some embodiments, the population of immune cells is a population of CD4+ and/or CD8+ T cells. In some embodiments, the percentage or amount of TCRαβ+ T cells is determined by subtracting the percentage or amount of CD3+/TCRγδ+ cells from the population of CD5+/CD3+ cells in step (b). In certain embodiments, the method further comprises the step of determining CD3+/TCRγδ+ cells. In some embodiments, the CAR T cells are allogeneic CAR T cells.

In a further aspect, the present disclosure provides a method of analyzing CAR T cells comprising the step of measuring surface CD107 of the CAR T cells after antigen stimulation, wherein an increased level of surface CD107 as compared to a level before antigen stimulation indicates polyfunctional CAR T cells. In some embodiments, the increased level of surface CD107 is an increased percentage or an increased mean/medium fluorescence intensities of surface CD107. In certain embodiments, the polyfunctional CAR T cells secret a higher level of TNFα after antigen stimulation as compared to CAR T cells that are not polyfunctional under the same conditions. In certain embodiments, the polyfunctional CAR T cells secret a higher level of IL2 after antigen stimulation as compared to CAR T cells that are not polyfunctional under the same conditions. In certain embodiments, the polyfunctional CAR T cells secret a higher level of IFNγ after antigen stimulation as compared to CAR T cells that are not polyfunctional under the same conditions. In some embodiments, the CAR T cells have been engineered to introduce one or more genetic modifications at the TCRα, and/or TCRβ locus to reduce or impair TCRαβ surface expression. In some embodiments, the one or more genetic modifications are at the TCRα, locus. In some embodiments, the method does not require a step of measuring one or more effector cytokines. In some embodiments, the method further comprises a step of measuring one or more cytokines selected from the group consisting of INFγ, TNFα, IL2, GM-CSF, CXCL1, IL-1b, IL-2, IL-4, IL-5, IL-6, IL-7, IL-8, IL-9, IL-10, IL-12, IL-17, IL-21, IL-22, IL-23, CXCL11, Mip1a, Mip1b, Mip3a, TNFb, Perforin, Granzyme A, Granzyme B, Granzyme H, CCL11, IP-10, CCL5, TGFb, sCD137, sCD40L, MCP-1, and MCP-4. In some embodiments, the CAR T cells is stimulated by co-culturing the CAR T cells with target cells expressing an antigen of the CAR. In some embodiments, wherein the target cells are tumor cells. In some embodiments, the level of surface CD107 is measured by flow cytometry. In some embodiments, the CD107 is CD107a and/or CD107b. In some embodiments, CD107 is measured about 2 hours, about 3 hours, about 4 hours, about 5 hours, about 6 hours, about 7 hours, about 8 hours, about 9 hours, or about 10 hours after the start of antigen activation. In some embodiments, CD107 is measured about 4-8 hours, about 4-6 hours, about 6-8 hours, about 6-10 hours, or about 8-10 hours after the start of antigen activation. In some embodiments, the CAR T cells are autologous CAR T cells. In some embodiments, the CAR T cells are allogeneic CAR T cells.

In some embodiments of any aspects of the disclosure, the method further comprises a step of measuring CAR+ T cells. In some embodiments, the percentage or amount of CAR+ T cells is measured using an anti-id antibody. In some embodiments, the population of immune cells are obtained from a healthy donor. In some embodiments, the CAR T cells are allogeneic CAR T cells.

In some embodiments, the immune cells express a CAR specific for EGFRvIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, BCMA, FLT3, CD70, DLL3, CD52 or CD34. In some embodiments, the CAR comprises an antigen binding domain that targets EGFRvIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, BCMA, FLT3, CD70, DLL3, CD52 or CD34.

In some embodiments, the method further comprises a step of filling the population of immune cells into one or more containers, if the amount or percentage of TCRαβ+ T cells is no more than a predetermined threshold and/or if the population of immune cells comprises polyfunctional CAR T cells optionally no less than a predetermined threshold, as determined herein. In some embodiments, the predetermined threshold for the percentage of TCRαβ+ T cells is about 3%, 2%, 1%, 0.9%, 0.8%, 0.7%, 0.6%, 0.5%, 0.4%, 0.3%, 0.2% or 0.1%. In some embodiments, the predetermined threshold for the percentage of polyfunctional CAR T cells is about 10%, 20%, 30%, 40%, 50%, 60%, 70%, 80% or 90% of the total population of CAR T cells.

In a further aspect, the present disclosure provides a method of preparing a cell-based drug product comprising engineered immune cells, said method comprising the method of analyzing a population of immune cells as described herein and the step of filling the population of immune cells into one or more containers, if the amount or percentage of TCRαβ+ T cells is no more than a predetermined threshold and/or if the population of immune cells comprises polyfunctional CAR T cells, optionally no less than a predetermined threshold.

In an additional aspect, the preset disclosure provides a kit or an article of manufacture for analyzing a CAR T cell comprising one or more reagents for the detection of CD3 and/or TCRγδ.

In some embodiments, the kit or article of manufacture further comprise one or more reagents for detecting CD45, CD5, CD52, CD107 (CD107a and/or CD107b), and/or a CAR. In some embodiments, the one or more reagents comprise an antibody, optionally conjugated with a detectable label. In some embodiments, the detectable label is selected from the group consisting of a fluorescent label, a photochromic compound, a proteinaceous fluorescent label, a magnetic label, a radiolabel, and a hapten. In some embodiments, the fluorescent label is selected from the group consisting of an Atto dye, an Alexafluor dye, quantum dots, Hydroxycoumarin, Aminocouramin, Methoxycourmarin, Cascade Blue, Pacific Blue, Pacific Orange, Lucifer Yellow, NBD, R-Phycoerythrin (PE), PE-Cy5 conjugates, PE-Cy7 conjugates, Red 613, PerCP, TruRed, FluorX, Fluorescein, BODIPY-FL, Cy2, Cy3, Cy3B, Cy3.5, Cy5, Cy5.5, Cy7, TRITC, X-Rhodamine, Lissamine Rhocamine B, Texas Red, Allophycocyanin (APC), APC-Cy7 conjugates, Indo-1, Fluo-3, Fluo-4, DCFH, DHR, SNARF, GFP (Y66H mutation), GFP (Y66F mutation), EBFP, EBFP2, Azurite, GFPuv, T-Sapphire, Cerulean, mCFP, mTurquoise2, ECFP, CyPet, GFP (Y66W mutation), mKeima-Red, TagCFP, AmCyan1, mTFP1, GFP (S65A mutation), Midorishi Cyan, Wild Type GFP, GFP (S65C mutation), TurboGFP, TagGFP, GFP (S65L mutation), Emerald, GFP (S65T mutation), EGFP, Azami Green, ZsGreenl, TagYFP, EYFP, Topaz, Venus, mCitrine, YPet, TurboYFP, ZsYellow1, Kusabira Orange, mOrange, Allophycocyanin (APC), mKO, TurboRFP, tdTomato, TagRFP, DsRed monomer, DsRed2 (“RFP”), mStrawberry, TurboFP602, AsRed2, mRFP1, J-Red, R-phycoerythrin (RPE), B-phycoeryhring (BPE), mCherry, HcRed1, Katusha, P3, Peridinin Chlorophyll (PerCP), mKate (TagFP635), TurboFP635, mPlum, and mRaspberry. In some embodiments, the one or more reagents are used for flow cytometry.

### BRIEF DESCRIPTION OF THE DRAWINGS

FIG. 1 shows quantitative results of flow cytometry analyses of residual % of TCRαβ+ cells gated by the TCRαβ cutoff set by donor cells determined pre-TCRαβ depletion from either donor 1 (ACC-1: assay control cells from donor 1) or donor 2 (ACC-2: assay control cells from donor 2). The results presented are triplicates with standard deviation (SD). See also Table 2. ALLO-501A: CD19 CAR T without a rituximab suicide switch.

FIGS. 2A and 2B depict panels of flow cytometry analysis showing the current gating in TCR ablated CAR T cells (FIG. 2A), and a correlation between CD3+ intracellular staining and CD5+ surface staining (FIG. 2B). See also Table 3.

FIG. 3 panels A-I show the workflow of sequential FACS analyses of CD19 CAR T cells for determining residual % of TCRab+ T cells after TCR ablation using an internal biological gate, and the level of CD107a after CD19 positive target cell activation.

FIG. 4 shows results of expression of various effector cytokines versus the expression of CAR in each population of CAR T cells, when the CAR T cells were coculture with CD19− KG1a cells, CD19+ Daudi cells or when the CAR T cells were incubated with PMA (phorbol myristate acetate) and Ionomycin (a calcium ionophore) as a positive control.

FIG. 5A shows correlation between CD107a and IFNg, IL2 and TNFα in each subset of CAR T cells either co-cultured with the CD19− KG1a cells or the CD19+ Daudi cells. FIGS. 5B-5D show results of further analysis of each cytokine in CAR T cells upon antigen activation: identification of CD5+/CD45+ cells (i.e., T cells, FIG. 5B); within the CD5+/CD45+ population, the delineation of CAR+ and TCRαβ+, IFNg+, IL2+, TNFα+, or CD107a+ staining (FIG. 5C); and within the CD5+/CD45+ population, CD107a staining coinciding with each of IFNg, 1L2 and TNFα (FIG. 5D).

FIG. 6 shows results analyzing the induction of individual or combination of effector cytokines (IL2, TNFα, and/or IFNg) from either CD107a+ or CD107a− CAR+ cells (connected by a line as shown in the graph) co-cultured with the CD19− KG1a cells or CD19+ Daudi cells. ALLO-501 and ALLO-501A: CD19 CAR T with or without the rituximab suicide switch, respectively.

FIGS. 7A-7B show correlations of surface expression of CD107a and intracellular staining of two or three cytokines, i.e., TNFα, 1L2 and IFNγ (FIG. 7A), or two cytokines, i.e., all combinations of two of TNFα, 1L2 and IFNγ (FIG. 7B) in anti-CD19 CAR T (ALLO-501A) or a non-CD19 CAR (CAR B) after 6 hrs co-culturing in the presence (solid symbol) or absence (open symbol) of respective target-positive cells.

### DETAILED DESCRIPTION

The instant disclosure relates to methods and reagents for analyzing engineered immune cells, including those comprising chimeric antigen receptors (CARs).

Provided herein are methods and reagents for analysis and/or characterization of engineered immune cells, including without limitation CAR T cells, for example allogeneic CAR T cells, for use in immunotherapy. The processes and reagents disclosed herein allow better characterization of CAR T cells in a manufacturing setting in compliance with cGMPs (current Good Manufacturing Practices). Also provided herein are processes, workflows, kits, articles of manufacture and reagents that allow reliable, scalable, and convenient analysis of critical attributes of CAR T drug products. When adopted as a set of standardized parameters and analytical procedures, the processes and/or reagents disclosed herein can further streamline commercial manufacturing processes, quality controls, and/or facilitate documentation for regulatory reviews.

As used herein, the terms “a” and “an” are used to mean one or more. For example, a reference to “a cell” or “an antibody” means “one or more cells” or “one or more antibodies.”

In one aspect, the present disclosure provides a method of analyzing or characterizing immune cells, especially allogeneic immune cells, such as allogeneic CAR T cells for use in immunotherapy. In particular, the present disclosure provides a method of analyzing a population of immune cells, wherein the population of immune cells has been engineered to introduce one or more genetic modifications at the TCRα and/or TCRβ locus to reduce or impair TCRαβ surface expression, the method comprising the steps of (a) obtaining or measuring viable CD45+ cells from the population of immune cells; (b) obtaining or measuring CD5+/CD3+ cells from the cells in step (a); and (c) measuring or determining a percentage or amount of CD3+/TCRγδ− cells from the cells in step (b), wherein the percentage or amount of CD3+/TCRγδ− cells in step (c) indicates a percentage or amount of residual TCRαβ+ T cells present in the population of immune cells.

The T cell receptor is an antigen receptor molecule that forms a complex on the surface of T cells, responsible for the recognition of antigen presented to T cells by MHC molecules, and the interaction of which may lead to the activation of the T cell and an immune response to the antigen. The human T cell receptor is a heterodimer consisting of two transmembrane heterodimeric glycoprotein chains, the α and β chains, each with two domains, which are linked by a disulfide bond. A small subset of T cells expresses the TCRγ6 complex instead. Due to the short cytoplasmic tail of the TCR, it cannot directly signal when it binds to a peptide-MHC complex. Instead the TCR is associated with a group of signaling molecules collectively called CD3 which transmit an intracellular signal when the TCR binds to a peptide-MHC complex. Interaction of TCRαβ on the surface of allogeneic T cells, i.e., T cells from a donor, with the host MHC molecules may lead to GvHD. Thus, one important strategy for allogeneic T cell therapy is to generate TCRαβ− T cells from an allogeneic source, such as for allogeneic CAR T cell therapy.

CD3 is made up of one γ and δ and two F molecules which all have in their extracellular domains some limited sequence homology to the immunoglobulin domain. These molecules have small cytoplasmic domains and transmembrane domains with negatively charged residues. In the membrane, these negatively charged residues form salt bridges with the positively charged residues in the transmembrane region of the TCR. The TCR-CD3 receptor complex is completed by two other invariant proteins ζ and η which form dimers linked by disulfide bonds. At the T cell surface, therefore, the TCR-CD3 complex is expressed as a αβ (or less commonly γδ) heterodimer, in association with CD3γε and CD3δε dimers with an intracellular ζζ homodimers or a ζη heterodimer. It is believed that if TCR expression is disrupted, CD3 surface expression may also be abolished.

As used herein, unless specifically indicated, the term “TCR+,” “TCR,” “TCR wildtype,” “TCRαβ wildtype,” “TCRαβ+,” or “TCRαβ” when used in reference to a cell or population of cells, including populations generated using the methods provided in the instant disclosure, refers to cells that express at least an endogenous TCRαβ heterodimer, although one or more components of the CD3 complex may or may not be expressed on the cell surface.

As used herein, the term “TCR−,” “TCR knockout,” or “TCRαβ−” when used in reference to a cell or population of cells, including populations generated using the methods provided in the instant disclosure, refers to cells that lack at least a TCRαβ heterodimer, although one or more components of the CD3 complex may or may not be expressed on the cell surface.

In some embodiments, the TCR− cells have been genetically modified, e.g., engineered to introduce one or more genetic modifications at the TCRα and/or TCRβ chain to reduce or impair the expression of the TCRαβ heterodimer on the cell surface. In some embodiments, the one or more genetic modifications are introduced at the TCRα chain constant region (TRAC). In some embodiments, the TCR− cells have been modified to reduce functional TCRαβ signaling. The modification can be genomic, e.g., genetic editing to disrupt or knock out at least a portion of the TCRα and/or TCRβ chain and thereby inactivate TCRαβ function, or epigenomic, e.g., using siRNA or other inhibitors to downregulate or abolish TCRαβ activities.

As used herein, the term “TCR-depleted” when used in reference to a population of cells generated using the methods provided in the instant disclosure, means a population of cells that comprises fewer cells expressing an endogenous TCRαβ heterodimer than a population of cells that is collected from a donor. For example, a population of TCR-depleted cells (or TCRαβ-depleted cells) can comprise 10%, 9%, 8%, 7%, 6%, 5%, 4%, 3%, 2%, 1% or less than 1% of cells expressing an endogenous TCRαβ complex.

### Characterization of CAR T Cells

### Determination of Remaining TCRαβ+ Cells in TCR− Allogeneic CAR T Products

Engineered allogeneic CAR-T products exemplify a strategy for generating next generation CAR-T therapeutics. However, the potential immune responses such as GvHD risk represents one of the significant safety or effectiveness concerns for allogenic CAR-T cell therapy. GvHD results from donor derived T cells recognizing HLA mismatch via TCRαβ and having the potential to attack the patient's tissues. GvHD can be serious and even fatal, even in the HLA matched donor setting, as minor mismatches can still provoke an immune response.

As described herein, the current disclosure provides methods for TCR− allogeneic CAR T cells by introducing one or more genetic modifications to the TCRα chain or TCRβ chain to reduce or impair the expression of TCRαβ complex on the cell surface. Any TCRαβ+ cells are removed in a process of TCRαβ+ cell depletion. Initially, engineered immune cells modified to be deficient for the endogenous TCRα and/or TCRβ gene are exposed to a TCR depletion reagent. In some embodiments, the TCR depletion reagent comprises an antibody targeting the TCRα polypeptide, TCRβ polypeptide, or TCRαβ heterodimer endogenously expressed on the surface of immune cells.

The anti-TCR antibody, or any other antibody can be conjugated, for example, to biotin to facilitate further labeling and/or separation using a secondary antibody (e.g., an anti-biotin antibody). The secondary antibody can be conjugated either directly or indirectly with a magnetic depletion reagent such as magnetic depletion agent such as magnetic microbeads (nanoparticles that are generally, but not necessarily, about 50 nm in diameter) or any other surface, such as an agarose bead, an acoustic wave particle, a plastic welled plate, a glass welled plate, a ceramic welled plate, a column, a cell culture bag, or a membrane. When magnetic microbeads are used, the microbeads facilitate separation of the TCR+ cells from the TCR− cells; when contacted with a magnetic column, the TCR+ cells can be retained on the column while unlabeled TCR− cells pass through to a collection bag. Acoustic wave particles can facilitate separation of TCR+ from the TCR− cells when exposed to an acoustic wave. While an anti-biotin antibody is provided in the context of the disclosed method, other biotin-binding partners such as streptavidin, avidin, and other proteins that recognize biotin can be employed in lieu of an anti-biotin antibody in all the methods provided herein.

In some embodiments, provided cells may be optionally sorted for other cell surface markers. For example, a subset of a population of immune cells can comprise engineered immune cells expressing an antigen-specific CAR, which itself comprises one or more epitopes specific for one or more monoclonal antibodies (e.g., exemplary mimotope sequences; see, e.g., WO2016/120216, incorporated by reference herein). In some embodiments, engineered immune cells expressing a CAR may be optionally sorted by using an anti-idiotypic antibody that binds to an epitope presented in the CAR extracellular antigen-binding domain. The method comprises contacting the population of immune cells with a monoclonal antibody specific for the epitopes and selecting the immune cells that bind to the monoclonal antibody to obtain a population of cells enriched in engineered immune cells expressing an antigen-specific CAR.

In some embodiments, said monoclonal antibody specific for said epitope is optionally conjugated to a fluorophore. In this embodiment, the step of sorting or selecting the cells that bind to the monoclonal antibody can be done by Fluorescence Activated Cell Sorting (FACS).

In some embodiments, said monoclonal antibody specific for said epitope is optionally conjugated to a magnetic particle. In this embodiment, the step of sorting or selecting the cells that bind to the monoclonal antibody can be done by Magnetic Activated Cell Sorting (MACS).

In some embodiments of the disclosed methods, sorting or separating TCR+ cells, optionally expressing a CAR, from TCR− cells can be achieved using Magnetic-Activated Cell Sorting (MACS). Magnetic-activated cell sorting is a method for separation of various cell populations depending on their surface antigens (CD molecules) by using superparamagnetic nanoparticles and columns. MACS can be used to obtain a very pure cell population. Cells in a single-cell suspension can be magnetically labeled with microbeads. The sample is applied to a column composed of a ferromagnetic material, which is covered with a coating not disruptive to cells, thus allowing fast and gentle separation of cells. The unlabeled cells pass through the column while the magnetically labeled cells are retained within the column. The flow-through can be collected as the unlabeled cell fraction. After a washing step, the column is removed from the separator, and the magnetically labeled cells are eluted from the column.

In some embodiments of the disclosed methods, sorting or separating TCR+ cells, optionally expressing a CAR, from TCR− cells can be achieved using acoustic wave separation in lieu of magnetic-based separation methods. While not wishing to be bound by theory, it is understood that acoustic wave separation relies on a three-dimensional standing wave to separate components of a mixture. As disclosed below, in some embodiments, the CAR T cells are also engineered to disrupt the expression of CD52 to facilitate the lymphodepletion process. In the context of the disclosed methods, an antibody such as an anti-TCR antibody and/or an anti-CD52 antibody can be conjugated to a surface, such as an acoustic wave particle. An acoustic wave particle can be a bead. In an embodiment, cells are exposed to acoustic wave particles bearing one or more of an anti-TCR antibody and/or an anti-CD52 antibody associating the acoustic wave particle with any cells expressing the target of interest. The cells are then placed in an acoustic chamber and exposed to an acoustic wave. Given the different properties of the bead-associated cells and cells that were not labeled with the antibody-bead particles, the acoustic wave separates the labeled and unlabeled cells, which can be collected while labeled cells (e.g., TCR+ or CD52+ cells) can be divert away from the unlabeled cells.

Current methods for removing residual TCR+ cells (TCRαβ+ cell depletion) such as the CliniMACS Prodigy® and TCR reagent kit, employs an anti-TCRαβ antibody to separate TCR+ cells (TCRαβ+ cells) from TCR− cells (TCRαβ− cells) using an anti-TCRαβ antibody and may achieve a 99% or greater TCR− cell purity in the final drug product (see, e.g., Radestad et al, (2014) J Immunol Res, Vol 2014: 578741). It is important to be able to confidently and reliably determine and quantify any residual levels of TCR+ cells after depletion in the final allogeneic CAR T drug product to understand any risk of GvHD associated with allogeneic CAR-T cell therapy, especially when used at high dose levels.

One of the challenges in accurately measuring residual TCR+ levels is to set an adequate cutoff/threshold (or gating) when analyzing the levels of TCRαβ, e.g., by flow cytometry. Allogeneic T cells from different donors may have different base line levels of surface TCRαβ expression when measured by a given method. It is therefore difficult to ascertain a universal cutoff or threshold of TCRαβ positivity that is applicable to all donor T cells: a cutoff or threshold that is set too high may result in under-reporting of TCRαβ+ events and potentially elevated risks to patients; a cutoff or threshold that is set too low may result in over-reporting of TCRαβ+ events and wasting TCR− allogeneic CAR T cells suitable for use in clinical studies or commercially. To account for the variability, unmodified T cells or source cells such as PBMC from each donor will have to be set aside and saved for later use for gate setting. The logistics can be complex and the process cumbersome especially in a large-scale manufacturing setting where different batches of CAR T cell products may be produced from different donors.

In addition, the lack of a compatible detection reagent may compound the problem. Using the same antibody for TCRαβ depletion and TCRαβ detection post depletion may contribute to inaccurate measurement and reporting of residual TCRαβ+ cells.

Accordingly, in one aspect, the instant disclosure provides a method of analyzing genetically modified allogeneic CAR T drug product, e.g., by measuring or determining any residual TCRαβ+ T cells present in the drug product, wherein the method does not require a donor-matched cutoff or threshold of the baseline level of TCRαβ expression, above which is considered TCRαβ positive. In some embodiments, the percentage or amount of residual TCRαβ+ T cells is determined without using a predetermined cutoff or threshold and/or without the need for a detection reagent compatible with the reagent for TCRαβ depletion. In some embodiments, the percentage or amount of residual TCRαβ+ cells can be determined using an internal biological control. In some embodiments, the percentage or amount of residual TCRαβ+ cells can be determined by accounting for T cells that are CD3+/TCRγδ+, wherein the T cells are identified based on surface expression of CD5.

Accordingly, in another aspect, the instant disclosure provides a method of detecting T cells by detecting surface expression of CD5. In some embodiments, the T cells do not express surface TCR, including TCRαβ and/or TCRγδ, and thus do not express or express much reduced levels of surface CD3. In some embodiments, the method provides a quantitative measurement of T cells that do not express surface TCR, including TCRαβ and/or TCRγδ, and thus do not express or express much reduced levels of surface CD3. In some embodiments, the method further comprising detecting surface expression of CD45+. In some embodiments, the T cells are genetically modified T cells.

Flow cytometry can be used to quantify cells expressing specific surface markers, such as TCRαβ, or quantifying cells of a specific cell type, within a population of cells. In general, flow cytometry is a method for quantifying components or structural features of cells primarily by optical means. Since different cell types can be distinguished by quantifying structural features, flow cytometry and cell sorting can be used to count and sort cells of different phenotypes in a mixture.

A flow cytometry analysis involves two primary steps: 1) labeling selected cell types with one or more detectable markers, and 2) determining the number of labeled cells relative to the total number of cells in the population. In some embodiments, the method of labeling cell types includes binding labeled antibodies to markers expressed by the specific cell type. The antibodies may be either directly labeled with a fluorescent compound or indirectly labeled using, for example, a fluorescent-labeled second antibody which recognizes the first antibody.

In some embodiments, provided cells may be subjected to further analysis for the expression of a CAR on the surface. The presence of the CAR may be detected by using an anti-id antibody specific for the antigen binding domain of the CAR. In some embodiments, the presence of the CAR may be detected by using an antibody specific for other portion of the CAR. In some embodiments, the presence of the CAR may be detected using the antigen that is specifically recognized and bound by the antigen binding domain of the CAR. In some embodiments, the antigen is directly or indirectly labeled for easy detection. In some embodiments, the CAR can be detected by extracellular staining or intracellular staining.

In some embodiments, the cells are analyzed for other surface markers indicative of different cell types in a population of cells, for example, effector cells, effector memory cells, central memory cells, stem central memory cells, etc. based on well-accepted specific surface markers for each cell type. In a further aspect, provided herein are methods of detecting surface markers indicative of other attributes of the CAR T cell product.

In some embodiments, provided cells may be subjected to further analysis for other surface markers. In some embodiments, the cells are analyzed for surface markers, the presence or absence of which may reflect the genetic modification(s) of the cells. For example, the cells have been genetically modified to knock out, in addition to TCR, the CD52 gene to acquire resistance to anti-CD52 Ab. In some embodiments, the anti-CD52 antibody is used as part of a lymphodepletion strategy as described below. Analysis of surface CD52 levels reflect the genetic knockout event in the CD52 locus of the cells. In some embodiments, the cells are analyzed for additional surface markers, the levels of which may indicate the potency or functionality of the cells. The analysis can be qualitative or quantitative.

Determination of Potency and/or Polyfunctional CAR T Cells

In this further aspect, the instant disclosure provides a method for analyzing and/or determining potency and/or polyfunctionality of an immune cell. In some embodiments, the immune cell is an engineered immune cell, for example, a CAR T cells. Currently, the method of evaluating the potency of CAR T products is rather limited. Upon exposing/binding to target cells, CAR T cells exert cytotoxicity partly through secretion of one or more effector cytokines. Effective cytokine induction can be used as an indication for potency or polyfunctionality of CAR T cells. Secreted cytokine can be measured by an immune assay such as ELISA. Cytokine induction of CAR T cells can also be assessed by intracellular staining after fixation of cells. However, both methods are cumbersome and neither method is amenable to highly streamlined GMP manufacturing process.

Potency, as used herein, can refer to the ability of one or more immune cells, such as CAR T cells, to kill a target cell, such as an antigen positive tumor cell.

Polyfunctionality, as used herein, can refer to the ability of one or more immune cells, such as CAR T cells, to secrete more than one effector cytokine or molecule upon target or antigen activation. In some embodiments, polyfunctional CAR T cells secret two or more effector cytokines, or three or more effector cytokines, upon target or antigen activation.

CD107, including CD107a (or lysosome-associated membrane protein (LAMP1)) and CD107b (LAMP2)) are major lysosomal membrane glycoproteins. The functions of CD107a and CD107b largely overlap. CD107 is released to the cell surface upon T cell activation and has been used as a T cell degranulation marker. T cell activation can also be measured by assessing elevated levels of individual effector cytokines, such as TNFα, I1L2, GM-CSF, and IFNγ, etc., by intracellular staining. See, e.g., Priceman et al., 2018, Oncoimmunology, 7:e1380764; and Kochenderfer et al., 2015, J. Clinical Oncology, 33:540. The steps of intracellular staining of these effector cytokines cannot be easily adapted into a GMP manufacturing process. The instant disclosure provides data demonstrating the correlation of CD107a surface expression and the induction of one or more, two or more, three or more, or a plurality of effector cytokines upon antigen activation. The correlation has not been shown previously in autologous or allogeneic CAR T cells, including TCR− allogeneic CAR T cells.

Thus, in this aspect, the instant disclosure provides a method of detecting elevated levels of surface expression of CD107 of CAR T cells upon exposing the CAR T cells to antigen or target cells, wherein the elevated levels of CD107 as compared to the level before antigen activation can be used as a proxy or indicator for potency or polyfunctionality of CAR T cells. In some embodiments, the elevated surface CD107 expression indicates the induction of one or more, two or more, or three or more effector cytokines, wherein the effector cytokines are INFγ, TNFα, IL2, GM-CSF, CXCL1, IL-1b, IL-2, IL-4, IL-5, IL-6, IL-7, IL-8, IL-9, IL-10, IL-12, IL-17, IL-21, IL-22, IL-23, CXCL11, Mip1a, Mip1b, Mip3a, TNFb, Perforin, Granzyme A, Granzyme B, Granzyme H, CCL11, IP-10, CCL5, TGFβ, sCD137, sCD40L, MCP-1, and/or MCP-4. In some embodiments, the effector cytokines are TNFα and IFNγ. In some embodiments, the effector cytokines are TNFα and IL2. In some embodiments, the effector cytokines are IFNγ and IL2. In some embodiments, the effector cytokines are TNFα, IFNγ and IL2. CD107a is most strongly correlated with IFNγ induction, which is a critical cytokine in eradicating tumor cells in the tumor microenvironment. In some embodiments, the instant disclosure provides a method of detecting elevated surface CD107 expression of CAR T cells upon antigen activation, wherein elevated levels of CD107 as compared to the levels before antigen activation demarcates target-specific induction of two or more of IFNγ, IL2, and/or TNFα across all CAR+ immune subsets. In some embodiments, the elevated level of CD107 is at least 1.5 fold, at least 2 fold, at least 3 fold, at least 4 fold, at least 5 fold, at least 6 fold, at least 7 fold, at least 8 fold, at least 9 fold, or at least 10 fold higher as compared to the level before antigen activation.

In some embodiments, the CAR T cells are autologous CAR T cells. In some embodiments, the CAR T cells are allogeneic CAR T cells.

In some embodiments, the CAR T cells are TCR− allogeneic CAR T cells. In contrast to TCR+ CAR T cells, in the absence of a functional TCR, the activation of the engineered T cells relies on the interaction of the CAR with the antigen. The correlation of elevated surface CD107 and the induction of effector cytokines has not been shown in CAR T cells without a functional TCRαβ complex. In some embodiments, the instant disclosure provides method of detecting elevated surface CD107 as an indicator for potency and/or polyfunctionality in TCR negative allogeneic CAR T cells.

The method of determining potency or polyfunctionality provided herein can be used for quality control of CAR T cells in a manufacturing process. In some embodiments, the instant disclosure provides method of manufacturing engineered immune cells, such as CAR T cells, that comprises the steps of producing the engineered immune cells, measuring an elevated level of surface CD107 of the engineered immune cells after antigen activation of the engineered immune cell drug substance, and, if the elevated level of surface CD107 reaches a threshold, filling the drug substance into a container or a vial to produce the drug product. In some embodiments, if the surface CD107 level does not meet a threshold, the downstream filling step is halted. In some embodiments, the threshold is a predetermined threshold. In some embodiments, the engineered immune cells are allogeneic CAR T cells. In some embodiments, the allogeneic CAR T cells are TCR− allogeneic CAR T cells. In some embodiments, the predetermined threshold is at least 1.5 fold, at least 2 fold, at least 3 fold, at least 4 fold, at least 5 fold, at least 6 fold, at least 7 fold, at least 8 fold, at least 9 fold, or at least 10 fold higher as compared to the level before antigen activation.

The measuring of surface CD107 expression by, e.g., flow cytometry, can be incorporated into a manufacturing quality control process with much ease, as compared to measuring individual effector cytokines intracellularly. In some embodiments, the method of detecting surface CD107 after antigen activation provided herein does not require the step of measuring one or more effector cytokines. In further embodiments, the method of analyzing potency and/or polyfunctionality of engineered immune cells, such as CAR T cells, by detecting surface CD107 after antigen activation does not require the step of measuring one or more effector cytokines.

The method provided herein can also be applied to analyzing samples from patients, who are undergoing cell therapies, such as CAR T cell therapy. In some embodiments, the samples are peripheral blood samples from patients, wherein the elevated levels of surface CD107 of CAR T cells after antigen activation indicate potency or polyfunctionality of the CAR T cell therapy. In some embodiments, the method further comprises a step of detecting the presence of CAR T cells in the sample. In some embodiments, the determination of surface CD107 after antigen activation can aid physicians in making dosing or treatment decisions.

Thus, the instant disclosure provides a method for measuring CD107 on the surface of CAR T cells after antigen stimulation, wherein an increased level of surface CD107 after antigen stimulation as compared to the level of surface CD107 before antigen stimulation indicates polyfunctional CAR T cells. In some embodiments, polyfunctional CAR T cells express increased levels of one or more, or two or more effector cytokines. In some embodiments, polyfunctional CAR T cells express increased levels of two or more effector cytokines.

Antigen stimulation (or antigen activation) of CAR T cells can be achieved by, for example, binding to antigens, binding to target cells, e.g., target tumor cells expressing the antigen, or by co-culturing with target cells, e.g., target tumor cells expressing the antigen. In some embodiments, the surface CD107 of CAR T cells is measured about 4 hours, about 5 hours, about 6 hours, about 7 hours, about 8 hours, about 9 hours, or about 10 hours after antigen activation. In some embodiments, the surface CD107 of CAR T cells is measured about 4 hours, about 5 hours, about 6 hours, about 7 hours, about 8 hours, about 9 hours, or about 10 hours after co-culturing with target cells. In some embodiments, the surface CD107 of CAR T cells is measured about 6 hours after co-culturing with target cells. In some embodiments, the CAR T cells are autologous CAR T cells. In some embodiments, the CAR T cells are allogeneic CAR T cells. In some embodiments, the CAR T cells are TCR− allogeneic CAR T cells. In some embodiments, the CD107 is CD107a (e.g., GenBank Accession No. NM_005561 or NCBI Gene ID #3916) and/or CD107b (e.g., GenBank Accession No. NM_001122606 or NCBI Gene ID #3920). In some embodiments, the CAR T cells are manufactured in compliance with cGMPs. In some embodiments, the one or more effector cytokines are INFg, TNFα, IL2, GM-CSF, CXCL1, IL-1b, IL-2, IL-4, IL-5, IL-6, IL-7, IL-8, IL-9, IL-10, IL-12, IL-17, IL-21, IL-22, IL-23, CXCL11, Mip1a, Mip1b, Mip3a, TNFb, Perforin, Granzyme A, Granzyme B, Granzyme H, CCL11, IP-10, CCL5, TGFb, sCD137, sCD40L, MCP-1, and/or MCP-4.

The instant disclosure also provides an overall process for characterizing and analyzing CAR T, especially TCR− allogeneic CAR T drug product. Accordingly, in one aspect, the instant disclosure provides a method for analyzing a population of engineered immune cells, for example, TCR− allogeneic CAR T cells, comprising steps of measuring or determining a percentage or amount of TCRαβ+ T cells in the population of immune cells, and measuring in the population of immune cells level of surface CD107 after antigen stimulation.

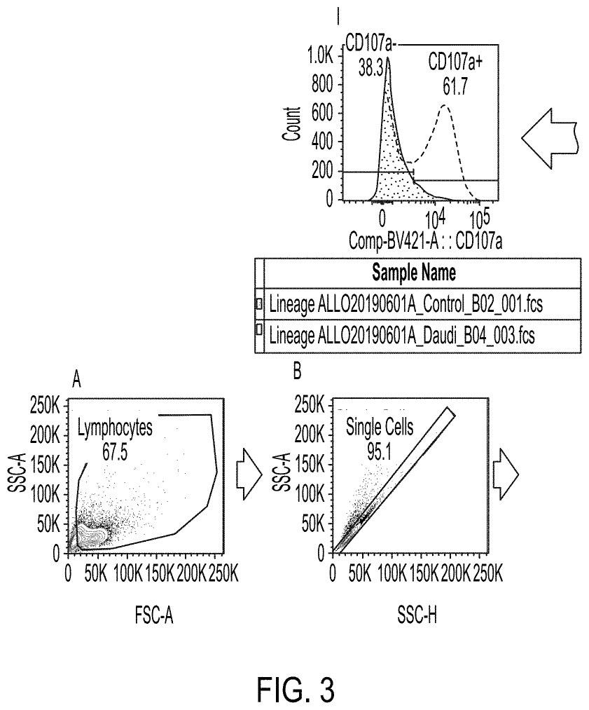

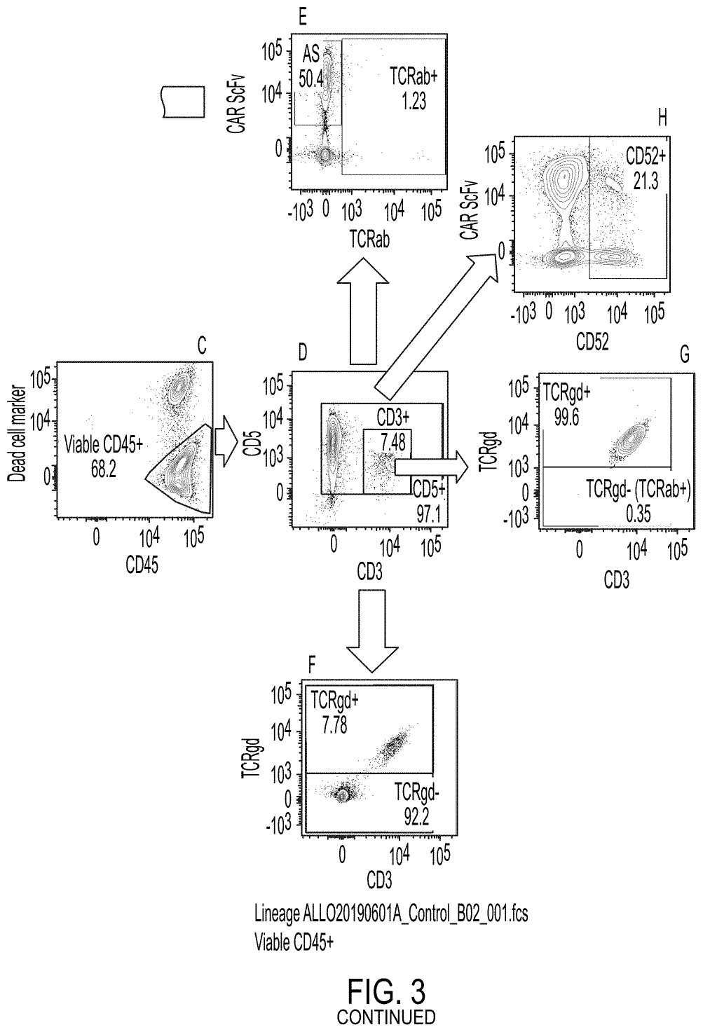

In some embodiments, the instant disclosure provides a method of analyzing a population of immune cells, wherein the population of immune cells has been engineered to introduce one or more genetic modifications at the TCRα, and/or TCRβ locus to reduce or impair TCRαβ surface expression, and wherein the population of lymphocytes has been engineered to express a chimeric antigen receptor (CAR), the method comprising the steps of (a) obtaining or measuring viable CD45+ cells from the population of immune cells; (b) obtaining or measuring CD5+/CD3+ cells from the cells in step a); (c) measuring or determining a percentage or amount of CD3+/TCRγδ− cells from the cells in step (b), wherein the percentage or amount of CD3+/TCRγδ− cells in step (c) indicates the percentage or amount of TCRαβ+ T cells in the population of immune cells; and (d) measuring in the population of immune cells (i) a percentage or amount of CAR+ T cells; and/or (ii) level of surface CD107 after antigen stimulation. In some embodiments, the workflow of the method can be illustrated as in FIG. 3.

In some embodiments, the method further comprises the step of measuring a percentage or amount of CAR+ T cells. In some embodiments, the percentage or amount of CAR+ T cells can be determined by using a reagent, for example, an anti-id antibody or an antigen. The antigen can be soluble or immobilized on a solid surface. The reagent can be directly labeled for detection or bound by a secondary labeled reagent for detection. In some embodiments, the method further comprises the step of measuring or detecting CD52+ cells. In some embodiments, the CAR T cells are manufactured in a GMP manufacturing process. In some embodiments, the population of engineered immune cells are TCR− allogeneic CAR T cells manufactured in a GMP manufacturing process. In some embodiments, the population of engineered immune cells are GMP allogeneic CAR T drug substance or drug product.

In certain embodiments, the CAR T cells are specific for EGFRvIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, BCMA, FLT3, CD70, DLL3, CD52 or CD34. In certain embodiments, the CAR T cells are EGFRvIII CAR T cells, CD19 CAR T cells, CD20 CAR T cells, CD33 CAR T cells, ROR1 CAR T cells, CD70 CAR T cells, FLT3 CAR T cells, BCMA CAR T cells, or DLL3 CAR T cells. In certain embodiments, the CAR T cells are CD19 CAR T cells. In certain embodiments, the CD19 CAR T cells comprise the C19 CAR comprising the sequence set forth in SEQ ID NO: 1 or SEQ ID NO:2.

(SEQ ID NO: 1)
EVQLQQSGPELIKPGASVKMSCKASGYTFTSYVMHWVKQKPGQGLEWI
GYINPYNDGTKYNEKFKGKATLTSDKSSSTAYMELSSLTSEDSAVYYC
ARGTYYYGSRVFDYWGQGTTLTVSSGGGGSGGGGSGGGGSDIVMTQAA
PSIPVTPGESVSISCRSSKSLLNSNGNTYLYWFLQRPGQSPQLLIYRM
SNLASGVPDRFSGSGSGTAFTLRISRVEAEDVGVYYCMQHLEYPFTFG
AGTKLELKRSDPTTTPAPRPPTPAPTIASQPLSLRPEACRPAAGGAVH
TRGLDFACDIYIWAPLAGTCGVLLLSLVITLYCKRGRKKLLYIFKQPF
MRPVQTTQEEDGCSCRFPEEEEGGCELRVKFSRSADAPAYQQGQNQLY
NELNLGRREEYDVLDKRRGRDPEMGGKPRRKNPQEGLYNELQKDKMAE
AYSEIGMKGERRRGKGHDGLYQGLSTATKDTYDALHMQALPPR
(SEQ ID NO: 2)
METDTLLLWVLLLWVPGSTGEVQLQQSGPELIKPGASVKMSCKASGYT
FTSYVMHWVKQKPGQGLEWIGYINPYNDGTKYNEKFKGKATLTSDKSS
STAYMELSSLTSEDSAVYYCARGTYYYGSRVFDYWGQGTTLTVSSGGG
GSGGGGSGGGGSDIVMTQAAPSIPVTPGESVSISCRSSKSLLNSNGNT
YLYWFLQRPGQSPQLLIYRMSNLASGVPDRFSGSGSGTAFTLRISRVE
AEDVGVYYCMQHLEYPFTFGAGTKLELKRSDPTTTPAPRPPTPAPTIA
SQPLSLRPEACRPAAGGAVHTRGLDFACDIYIWAPLAGTCGVLLLSLV
ITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKP
RRKNPQEGLYNELQKDKMAEAYSEIGMKGERRRGKGHDGLYQGLSTAT
KDTYDALHMQALPPR

### 1. Immune Cells

Cells suitable for use with the methods and/or reagents described herein include immune cells.

Prior to the in vitro manipulation or genetic modification (e.g., as described herein), cells for use in methods described herein (e.g., immune cells) can be obtained from a subject. Cells can be obtained from a number of non-limiting sources, including peripheral blood mononuclear cells, bone marrow, lymph node tissue, cord blood, thymus tissue, stem cell- or iPSC-derived immune cells, tissue from a site of infection, ascites, pleural effusion, spleen tissue, and tumors. In some embodiments, any number of T cell lines available and known to those skilled in the art, can be used. In some embodiments, cells can be derived from a healthy donor, from a patient diagnosed with cancer or from a patient diagnosed with an infection. In some embodiments, cells can be part of a mixed population of cells which present different phenotypic characteristics.

In some embodiments, immune cells are autologous immune cells obtained from a subject who will ultimately receive the engineered immune cells. In some embodiments, immune cells are allogeneic immune cells obtained from a donor, who is a different individual from the subject who will receive the engineered immune cells.

In some embodiments, immune cells comprise T cells. T cells can be obtained from a number of sources, including peripheral blood mononuclear cells (PBMCs), bone marrow, lymph nodes tissue, cord blood, thymus tissue, stem cell- or iPSC-derived T cells, tissue from a site of infection, ascites, pleural effusion, spleen tissue, and tumors. In certain some embodiments, T cells can be obtained from a volume of blood collected from the subject using any number of techniques known to the skilled person, such as FICOLL™ separation.

Cells can be obtained from the circulating blood of an individual by apheresis. The apheresis product typically contains lymphocytes, including T cells, monocytes, granulocytes, B cells, other nucleated white blood cells, red blood cells, and platelets. In certain some embodiments, the cells collected by apheresis can be washed to remove the plasma fraction, and placed in an appropriate buffer or media for subsequent processing.

PBMCs can be used directly for genetic modification with the immune cells (such as CARs or TCRs) using methods as described herein. In certain embodiments, after isolating the PBMCs, T lymphocytes can be further isolated and both cytotoxic and helper T lymphocytes can be sorted into naive, memory, and effector T cell subpopulations either before or after genetic modification and/or expansion.

In certain embodiments, T cells are isolated from PBMCs by lysing the red blood cells and depleting the monocytes, for example, using centrifugation through a PERCOLL™ gradient. A specific subpopulation of T cells, such as CCR7+, CD95+, CD122, CD27+, CD69+, CD127+, CD28+, CD3+, CD4+, CD8+, CD25+, CD62L+, CD45RA+, and CD45RO+ T cells can be further isolated by positive or negative selection techniques known in the art. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. Flow cytometry and cell sorting can also be used to isolate cell populations of interest for use in the present disclosure.

In some embodiments, a population of T cells is enriched for CD4+ cells.

In some embodiments, a population of T cells is enriched for CD8+ cells.

In some embodiments, CD8+ cells are further sorted into naive, central memory, and effector cells by identifying cell surface antigens that are associated with each of these types of cells. In some embodiments the expression of phenotypic markers for naïve T cells include CD45RA+, CD95−, IL2R−, CCR7+, and CD62L+. In some embodiments the expression of phenotypic markers for stem cell memory T cells include CD45RA+, CD95+, IL2Rβ+, CCR7+, and CD62L+. In some embodiments the expression of phenotypic markers for central memory T cells include CD45RO+, CD95+, IL2Rβ+, CCR7+, and CD62L+. In some embodiments the expression of phenotypic markers for effector memory T cells include CD45RO+, CD95+, IL2Rβ+, CCR7−, and CD62L−. In some embodiments the expression of phenotypic markers for T effector cells include CD45RA+, CD95+, IL2Rβ+, CCR7−, and CD62L−. Thus, CD4+ and/or CD8+ T helper cells can be sorted into naive, stem cell memory, central memory, effector memory and T effector cells by identifying cell populations that have cell surface antigens.

It will be appreciated that PBMCs can further include other cytotoxic lymphocytes such as NK cells or NKT cells. An expression vector carrying the coding sequence of a chimeric receptor as disclosed herein can be introduced into a population of human donor T cells, NK cells or NKT cells. Standard procedures are used for cryopreservation of T cells expressing the CAR for storage and/or preparation for use in a human subject. In one embodiment, the in vitro transduction, culture and/or expansion of T cells are performed in the absence of non-human animal derived products such as fetal calf serum and fetal bovine serum. In various embodiments a cryopreservative media can comprise, for example, CryoStor® CS2, CS5, or CS10 or other medium comprising DMSO, or a medium that does not comprise DMSO.

### 2. Engineered Immune Cells

Provided herein are engineered immune cells expressing the CARs of the disclosure (e.g., CAR-T cells).

In some embodiments, an engineered immune cell comprises a population of CARs, each CAR comprising extracellular antigen-binding domains. In some embodiments, an engineered immune cell comprises a population of CARs, each CAR comprising different extracellular antigen-binding domains. In some embodiments, an immune cell comprises a population of CARs, each CAR comprising the same extracellular antigen-binding domains.

The engineered immune cells can be allogeneic or autologous.

In some embodiments, the engineered immune cell is a T cell (e.g., inflammatory T-lymphocyte cytotoxic T-lymphocyte, regulatory T-lymphocyte, helper T-lymphocyte, tumor infiltrating lymphocyte (TIL)), NK cell, NK-T-cell, TCR-expressing cell, dendritic cell, killer dendritic cell, a mast cell, or a B-cell. In some embodiments, the cell can be derived from the group consisting of CD4+ T-lymphocytes and CD8+ T-lymphocytes. In some exemplary embodiments, the engineered immune cell is a T cell. In some exemplary embodiments, the engineered immune cell is an alpha beta T cell. In some exemplary embodiments, the engineered immune cell is a gamma delta T cell. In some exemplary embodiments, the engineered immune cell is a macrophage.

In some embodiments, the engineered immune cell can be derived from, for example without limitation, a stem cell. The stem cells can be adult stem cells, non-human embryonic stem cells, more particularly non-human stem cells, cord blood stem cells, progenitor cells, bone marrow stem cells, induced pluripotent stem cells (iPSC), totipotent stem cells or hematopoietic stem cells. Stem cells can be CD34+ or CD34−.

In some embodiments, the cell is obtained or prepared from peripheral blood. In some embodiments, the cell is obtained or prepared from peripheral blood mononuclear cells (PBMCs). In some embodiments, the cell is obtained or prepared from bone marrow. In some embodiments, the cell is obtained or prepared from umbilical cord blood. In some embodiments, the cell is a human cell. In some embodiments, the cell is transfected or transduced by the nucleic acid vector using a method selected from the group consisting of electroporation, sonoporation, biolistics (e.g., Gene Gun), transfection, lipid transfection, polymer transfection, nanoparticles, viral transduction or viral transfection (e.g., retrovirus, lentivirus, AAV) or polyplexes. In some embodiments the cell is a T cell that has been reprogrammed from a non-T cell. In some embodiments the cell is a T cell that has been reprogrammed from a T cell.

### Binding Agents (Including Antibodies and Fragments Thereof)

In embodiments, the disclosed methods comprise the use of an antibody or antigen binding agent (e.g., comprising an antigen binding domain or comprising an antibody or fragment thereof). As discussed below, in various embodiments engineered immune cells can also comprise a binding agent.

As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CHI, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain. Those skilled in the art are well familiar with antibody structure and sequence elements, recognize “variable” and “constant” regions in provided sequences, and understand that there may be some flexibility in definition of a “boundary” between such domains such that different presentations of the same antibody chain sequence may, for example, indicate such a boundary at a location that is shifted one or a few residues relative to a different presentation of the same antibody chain sequence.

Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation.

For purposes of the instant disclosure, in certain embodiments, any polypeptide or complex of polypeptides that includes sufficient immunoglobulin domain sequences as found in natural antibodies can be referred to and/or used as an “antibody,” whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art.

Moreover, the term “antibody” as used herein, can refer to any of the art-known or developed constructs or formats for utilizing antibody structural and functional features in alternative presentation. For example, in some embodiments, an antibody utilized in the methods of the instant disclosure is in a format selected from, but not limited to, intact IgA, IgG, IgE or IgM antibodies; bi- or multi-specific antibodies (e.g., Zybodies®, etc.); antibody fragments such as Fab fragments, Fab fragments, F(ab)2 fragments, Fd fragments, and isolated CDRs or sets thereof; single chain variable fragments (scFVs); polypeptide-Fc fusions; single domain antibodies (e.g., shark single domain antibodies such as IgNAR or fragments thereof); camelid antibodies (also referred to herein as nanobodies or VHHs); shark antibodies, masked antibodies (e.g., Probodies®); Small Modular ImmunoPharmaceuticals (SMIPs™); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc.), or other pendant group (e.g., poly-ethylene glycol, etc.).

As used herein, the term “antibody agent” generally refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc. as is known in the art. In many embodiments, the term “antibody agent” is used to refer to one or more of the art-known or developed constructs or formats for utilizing antibody structural and functional features in alternative presentation. For example, an antibody agent utilized in accordance with the present invention is in a format selected from, but not limited to, intact IgA, IgG, IgE or IgM antibodies; bi- or multi-specific antibodies (e.g., Zybodies®, etc.); antibody fragments such as Fab fragments, Fab′ fragments, F(ab′)2 fragments, Fd fragments, and isolated CDRs or sets thereof, single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (SMIPs™); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s.

An antibody or antibody agent used in performing the methods of the instant disclosure can be single chained or double chained. In some embodiments, the antibody or antigen binding molecule is single chained. In certain embodiments, the antigen binding molecule is selected from the group consisting of an scFv, a Fab, a Fab′, a Fv, a F(ab′)₂, a dAb, and any combination thereof.

Antibodies and antibody agents include antibody fragments. An antibody fragment comprises a portion of an intact antibody, such as the antigen binding or variable region of the intact antibody. Antibody fragments include, but are not limited to, Fab, Fab′, Fab′-SH, F(ab′)₂, Fv, diabody, linear antibodies, multispecific formed from antibody fragments antibodies and scFv fragments, and other fragments. Antibodies also include, but are not limited to, polyclonal monoclonal, chimeric dAb (domain antibody), single chain, Fab, Fa, F(ab′)₂ fragments, and scFvs. An antibody can be a whole antibody, or immunoglobulin, or an antibody fragment. Antibody fragments can be made by various techniques, including but not limited to proteolytic digestion of an intact antibody as well as production by recombinant host cells (e.g., E. coli, Chinese Hamster Ovary (CHO) cells, or phage), as known in the art.

In some embodiments, an antibody or antibody agent can be a chimeric antibody (see, e.g., U.S. Pat. No. 4,816,567; and Morrison et al., Proc. Natl. Acad. Sci. USA, 81:6851-6855 (1984)). A chimeric antibody can be an antibody in which a portion of the heavy and/or light chain is derived from a particular source or species, while the remainder of the heavy and/or light chain is derived from a different source or species. In one example, a chimeric antibody can comprise a non-human variable region (e.g., a variable region derived from a mouse, rat, hamster, rabbit, or non-human primate, such as a monkey) and a human constant region. In a further example, a chimeric antibody can be a “class switched” antibody in which the class or subclass has been changed from that of the parent antibody. Chimeric antibodies include antigen-binding fragments thereof.

In some embodiments, a chimeric antibody can be a humanized antibody (See, e.g., Almagro and Fransson, Front. Biosci., 13:1619-1633 (2008); Riechmann et al., Nature, 332:323-329 (1988); Queen et al., Proc. Natl Acad. Sci. USA 86:10029-10033 (1989); U.S. Pat. Nos. 5,821,337, 7,527,791, 6,982,321, and 7,087,409; Kashmiri et al., Methods 36:25-34 (2005); Padlan, Mol. Immunol, 28:489-498 (1991); Dall'Acqua et al., Methods, 36:43-60 (2005); Osbourn et al., Methods, 36:61-68 (2005); and Klimka et al., Br. J. Cancer, 83:252-260 (2000)). A humanized antibody is a chimeric antibody comprising amino acid residues from non-human hypervariable regions and amino acid residues from human FRs. In certain embodiments, a humanized antibody will comprise substantially all of at least one, and typically two, variable domains, in which all or substantially all of the hypervariable regions (e.g., CDRs) correspond to those of a non-human antibody, and all or substantially all of the Framework Regions (FRs) correspond to those of a human antibody. A humanized antibody optionally can comprise at least a portion of an antibody constant region derived from a human antibody.

In some embodiments, an antibody or antibody agent provided herein is a human antibody. Human antibodies can be produced using various techniques known in the art (See, e.g., van Dijk and van de Winkel, Curr. Opin. Pharmacol, 5: 368-74 (2001); and Lonberg, Curr. Opin. Immunol, 20:450-459 (2008)). A human antibody can be one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies may be prepared using methods well known in the art.

### Chimeric Antigen Receptors

As used herein, chimeric antigen receptors (CARs) are proteins that specifically recognize target antigens (e.g., target antigens on cancer cells). When bound to the target antigen, the CAR can activate the immune cell to attack and destroy the cell bearing that antigen (e.g., the cancer cell). CARs can also incorporate costimulatory or signaling domains to increase their potency. See Krause et al., J. Exp. Med., Volume 188, No. 4, 1998 (619-626); Finney et al., Journal of Immunology, 1998, 161: 2791-2797, Song et al., Blood 119:696-706 (2012); Kalos et al., Sci. Transl. Med. 3:95 (2011); Porter et al., N. Engl. J. Med. 365:725-33 (2011), and Gross et al., Annu. Rev. Pharmacol. Toxicol. 56:59-83 (2016); U.S. Pat. Nos. 7,741,465, and 6,319,494.

Chimeric antigen receptors described herein comprise an extracellular domain, a transmembrane domain, and an intracellular domain, wherein the extracellular domain comprises an antigen binding domain that specifically binds to the target.

In some embodiments, antigen-specific CARs further comprise a safety switch and/or one or more monoclonal antibody specific-epitope.

i. Antigen Binding Domains

As discussed above, CARs described herein comprise an antigen binding domain. An “antigen binding domain” as used herein means any polypeptide that binds a specified target antigen. In some embodiments, the antigen binding domain binds to an antigen on a tumor cell. In some embodiments, the antigen binding domain binds to an antigen on a cell involved in a hyperproliferative disease.

In some embodiments, the antigen binding domain comprises a variable heavy chain, variable light chain, and/or one or more CDRs described herein. In some embodiments, the antigen binding domain is a single chain variable fragment (scFv), comprising light chain CDRs CDR1, CDR2 and CDR3, and heavy chain CDRs CDR1, CDR2 and CDR3.

An antigen binding domain is said to be “selective” when it binds to one target more tightly or with higher affinity than it binds to a second target.

The antigen binding domain of the CAR selectively targets a cancer antigen. In some embodiments, the cancer antigen is selected from EGFRvIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, BCMA, FLT3, CD70, DLL3, CD52 or CD34. In some embodiments, the CAR comprises an antigen binding domain that targets EGFRvIII, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, BCMA, FLT3, CD70, DLL3, CD52 or CD34.

In some embodiments, the cancer antigen is selected from the group consisting of carbonic anhydrase IX (CAIX), carcinoembryonic antigen (CEA), CDS, CD7, CDIO, CD19, CD20, CD22, CD30, CD33, CD34, CD38, CD41, CD44, CD49f, CD56, CD74, CD123, CD133, CD138, an antigen of a cytomegalovirus (CMV) infected cell (e.g., a cell surface antigen), epithelial glycoprotein (EGP 2), epithelial glycoprotein-40 (EGP-40), epithelial cell adhesion molecule (EpCAM), receptor tyrosine-protein kinases erb-B2,3,4, folate-binding protein (FBP), fetal acetylcholine receptor (AChR), folate receptors, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Ra2), κ-light chain, kinase insert domain receptor (KDR), Lewis A (CA19.9), LI cell adhesion molecule (LICAM), melanoma antigen family A, 1 (MAGE-AI), Mucin 16 (Muc-16), Mucin 1 (Muc-1), Mesothelin (MSLN), NKG2D ligands, cancer-testis antigen NY-ESO-1, oncofetal antigen (h5T4), prostate stem cell antigen (PSCA), prostate-specific membrane antigen (PSMA), tumor-associated glycoprotein 72 (TAG-72), vascular endothelial growth factor R2 (VEGF-R2), and Wilms tumor protein (WT-1).

Variants of the antigen binding domains (e.g., variants of the CDRs, VH and/or VL) are also within the scope of the disclosure, e.g., variable light and/or variable heavy chains that each have at least 70-80%, 80-85%, 85-90%, 90-95%, 95-97%, 97-99%, or above 99% identity to the amino acid sequences of antigen binding domain sequences. In some instances, such molecules include at least one heavy chain and one light chain, whereas in other instances the variant forms contain two variable light chains and two variable heavy chains (or subparts thereof). A skilled artisan will be able to determine suitable variants of the antigen binding domains as set forth herein using well-known techniques. In certain embodiments, one skilled in the art can identify suitable areas of the molecule that can be changed without destroying activity by targeting regions not believed to be important for activity.

In certain some embodiments, the polypeptide structure of the antigen binding domains is based on antibodies, including, but not limited to, monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), and fragments thereof, respectively. In some embodiments, the antigen binding domain comprises or consists of avimers.

In some embodiments, an antigen binding domain is a scFv.

In some embodiments, an antigen-selective CAR comprises a leader or signal peptide

In other embodiments, the disclosure relates to isolated polynucleotides encoding any one of the antigen binding domains described herein. In some embodiments, the disclosure relates to isolated polynucleotides encoding a CAR. Also provided herein are vectors comprising the polynucleotides, and methods of making same.

In other embodiments, the disclosure relates to isolated polynucleotides encoding any one of the antigen binding domains described herein. In some embodiments, the disclosure relates to isolated polynucleotides encoding a CAR. Also provided herein are vectors comprising the polynucleotides, and methods of making same.

In some embodiments, a CAR-immune cell (e.g., CAR-T cell) which can form a component of a population of cells generated by practicing the methods of the instant disclosure comprises a polynucleotide encoding a safety switch polypeptide, such as for example RQR8. See, e.g., WO2013153391A, which is hereby incorporated by reference in its entirety. In a CAR-immune cell (e.g., a CAR-T cell) comprising the polynucleotide, the safety switch polypeptide can be expressed at the surface of a CAR-immune cell (e.g., CAR-T cell).

ii. Hinge Domain

The extracellular domain of the CARs of the disclosure can comprise a “hinge” domain (or hinge region). The term generally refers to any polypeptide that functions to link the transmembrane domain in a CAR to the extracellular antigen binding domain in a CAR. In particular, hinge domains can be used to provide more flexibility and accessibility for the extracellular antigen binding domain.

A hinge domain can comprise up to 300 amino acids—in some embodiments 10 to 100 amino acids or in some embodiments 25 to 50 amino acids. The hinge domain can be derived from all or part of naturally occurring molecules, such as from all or part of the extracellular region of CD8, CD4, CD28, 4-1BB, or IgG (in particular, the hinge region of an IgG; it will be appreciated that the hinge region can contain some or all of a member of the immunoglobulin family such as IgG1, IgG2, IgG3, IgG4, IgA, IgD, IgE, IgM, or fragment thereof), or from all or part of an antibody heavy-chain constant region. Alternatively, the hinge domain can be a synthetic sequence that corresponds to a naturally occurring hinge sequence, or can be an entirely synthetic hinge sequence. In some embodiments said hinge domain is a part of human CD8α chain (e.g., NP_001139345.1). In other embodiments, said hinge and transmembrane domains comprise a part of human CD8α chain. In some embodiments, the hinge domain of CARs described herein comprises a subsequence of CD8α, an IgG1, IgG4, PD-1 or an FcγRIIIα, in particular the hinge region of any of an CD8α, an IgG1, IgG4, PD-1 or an FcγRIIIα. In some embodiments, the hinge domain comprises a human CD8α hinge, a human IgG1 hinge, a human IgG4, a human PD-1 or a human FcγRIIIα hinge. In some embodiments the CARs disclosed herein comprise a scFv, CD8α human hinge and transmembrane domains, the CD3ζ signaling domain, and 4-1BB signaling domain.

iii. Transmembrane Domain

The CARs of the disclosure are designed with a transmembrane domain that is fused to the extracellular domain of the CAR. It can similarly be fused to the intracellular domain of the CAR. In some instances, the transmembrane domain can be selected or modified by amino acid substitution to avoid binding of such domains to the transmembrane domains of the same or different surface membrane proteins to minimize interactions with other members of the receptor complex. In some embodiments, short linkers can form linkages between any or some of the extracellular, transmembrane, and intracellular domains of the CAR.

Suitable transmembrane domains for a CAR disclosed herein have the ability to (a) be expressed at the surface an immune cell such as, for example without limitation, a lymphocyte cell, such as a T helper (Tₕ) cell, cytotoxic T (T_{c}) cell, T regulatory (T_{reg}) cell, or Natural killer (NK) cells, and/or (b) interact with the extracellular antigen binding domain and intracellular signaling domain for directing the cellular response of an immune cell against a target cell.

The transmembrane domain can be derived either from a natural or from a synthetic source. Where the source is natural, the domain can be derived from any membrane-bound or transmembrane protein.

Transmembrane regions of particular use in this disclosure can be derived from (comprise, or correspond to) CD28, OX-40, 4-1BB/CD137, CD2, CD7, CD27, CD30, CD40, programmed death-1 (PD-1), inducible T cell costimulator (ICOS), lymphocyte function-associated antigen-1 (LFA-1, CD1-1a/CD18), CD3 gamma, CD3 delta, CD3 epsilon, CD247, CD276 (B7-H3), LIGHT, (TNFSFi4), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptors, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD1 ld, ITGAE, CD103, ITGAL, CD1 la, LFA-1, ITGAM, CD1 lb, ITGAX, CD1 lc, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

As non-limiting examples, the transmembrane region can be derived from, or be a portion of a T cell receptor such as α, β, γ or δ, polypeptide constituting CD3 complex, IL-2 receptor p55 (α chain), p75 (β chain) or γ chain, subunit chain of Fc receptors, in particular Fcγ receptor III or CD proteins. Alternatively, the transmembrane domain can be synthetic and can comprise predominantly hydrophobic residues such as leucine and valine. In some embodiments said transmembrane domain is derived from the human CD8α chain (e.g., NP_001139345.1).

In some embodiments, the transmembrane domain in the CAR of the disclosure is a CD8α transmembrane domain.

In some embodiments, the transmembrane domain in the CAR of the disclosure is a CD28 transmembrane domain.

iv. Intracellular Domain

The intracellular (cytoplasmic) domain of the CARs of the disclosure can provide activation of at least one of the normal effector functions of the immune cell comprising the CAR. Effector function of a T cell, for example, can refer to cytolytic activity or helper activity, including the secretion of cytokines.

In some embodiments, an activating intracellular signaling domain for use in a CAR can be the cytoplasmic sequences of, for example without limitation, the T cell receptor and co-receptors that act in concert to initiate signal transduction following antigen receptor engagement, as well as any derivative or variant of these sequences and any synthetic sequence that has the same functional capability.

It will be appreciated that suitable (e.g., activating) intracellular domains include, but are not limited to signaling domains derived from (or corresponding to) CD28, OX-40, 4-1BB/CD137, CD2, CD7, CD27, CD30, CD40, programmed death-1 (PD-1), inducible T cell costimulator (ICOS), lymphocyte function-associated antigen-1 (LFA-1, CD1-la/CD18), CD3 gamma, CD3 delta, CD3 epsilon, CD247, CD276 (B7-H3), LIGHT, (TNFSFi4), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptors, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD1 ld, ITGAE, CD103, ITGAL, CD1 la, LFA-1, ITGAM, CD1 lb, ITGAX, CD1 lc, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

The intracellular domains of the CARs of the disclosure can incorporate, in addition to the activating domains described above, co-stimulatory signaling domains (interchangeably referred to herein as costimulatory molecules) to increase their potency. Costimulatory domains can provide a signal in addition to the primary signal provided by an activating molecule as described herein.

It will be appreciated that suitable costimulatory domains within the scope of the disclosure can be derived from (or correspond to) for example, CD28, OX40, 4-1BB/CD137, CD2, CD3 (alpha, beta, delta, epsilon, gamma, zeta), CD4, CD5, CD7, CD9, CD16, CD22, CD27, CD30, CD 33, CD37, CD40, CD 45, CD64, CD80, CD86, CD134, CD137, CD154, PD-1, ICOS, lymphocyte function-associated antigen-1 (LFA-1 (CD1 la/CD18), CD247, CD276 (B7-H3), LIGHT (tumor necrosis factor superfamily member 14; TNFSF14), NKG2C, Ig alpha (CD79a), DAP-10, Fc gamma receptor, MHC class I molecule, TNFR, integrin, signaling lymphocytic activation molecule, BTLA, Toll ligand receptors, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD1-1d, ITGAE, CD103, ITGAL, CD1-1a, LFA-1, ITGAM, CD1-lb, ITGAX, CD1-lc, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD83 ligand, or fragments or combinations thereof. It will be appreciated that additional costimulatory molecules, or fragments thereof, not listed above are within the scope of the disclosure.

In some embodiments, the intracellular/cytoplasmic domain of the CAR can be designed to comprise the 4-1BB/CD137 domain by itself or combined with any other desired intracellular domain(s) useful in the context of the CAR of the disclosure. The complete native amino acid sequence of 4-1BB/CD137 is described in NCBI Reference Sequence: NP_001552.2. The complete native 4-1BB/CD137 nucleic acid sequence is described in NCBI Reference Sequence: NM_001561.5.

In some embodiments, the intracellular/cytoplasmic domain of the CAR can be designed to comprise the CD28 domain by itself or combined with any other desired intracellular domain(s) useful in the context of the CAR of the disclosure. The complete native amino acid sequence of CD28 is described in NCBI Reference Sequence: NP_006130.1. The complete native CD28 nucleic acid sequence is described in NCBI Reference Sequence: NM_006139.1.

In some embodiments, the intracellular/cytoplasmic domain of the CAR can be designed to comprise the CD3 zeta domain by itself or combined with any other desired intracellular domain(s) useful in the context of the CAR of the disclosure.

For example, the intracellular domain of the CAR can comprise a CD3 zeta chain portion and a portion of a costimulatory signaling molecule. The intracellular signaling sequences within the intracellular signaling portion of the CAR of the disclosure can be linked to each other in a random or specified order. In some embodiments, the intracellular domain is designed to comprise the activating domain of CD3 zeta and a signaling domain of CD28. In some embodiments, the intracellular domain is designed to comprise the activating domain of CD3 zeta and a signaling domain of 4-1BB.

In some embodiments the intracellular signaling domain of the CAR of the disclosure comprises a domain of a co-stimulatory molecule. In some embodiments, the intracellular signaling domain of a CAR of the disclosure comprises a part of co-stimulatory molecule selected from the group consisting of fragment of 4-1BB (GenBank: AAA53133.) and CD28 (NP_006130.1).

### Engineered Immune Cells Comprising CARs

Also provided herein are engineered immune cells and populations of engineered immune cells expressing CAR (e.g., CAR-T cells or CAR+ cells), which are depleted of cells expressing endogenous TCR.

In some embodiments, an engineered immune cell comprises a CAR T cell, each CAR T cell comprising an extracellular antigen-binding domain and has reduced or eliminated expression of endogenous TCR. In some embodiments, a population of engineered immune cells comprises a population of CAR T cells, each CAR T cell comprising two or more different extracellular antigen-binding domain and has reduced or eliminated expression of endogenous TCR. In some embodiments, an immune cell comprises a population of CARs, each CAR T cell comprising the same extracellular antigen-binding domains and has reduced or eliminated expression of endogenous TCR.

The engineered immune cells can be allogeneic or autologous.

In some embodiments, an engineered immune cell or population of engineered immune cells is a T cell (e.g., inflammatory T-lymphocyte cytotoxic T-lymphocyte, regulatory T-lymphocyte, helper T-lymphocyte, tumor infiltrating lymphocyte (TIL)), NK cell, NK-T-cell, TCR-expressing cell, dendritic cell, killer dendritic cell, a mast cell, or a B-cell, and expresses a CAR. In some embodiments, the T cell can be derived from the group consisting of CD4+ T lymphocytes, CD8+ T lymphocytes or population comprising a combination of CD4+ and CD8+ T cells.

In some embodiments, an engineered immune cell or population of engineered immune cells that are generated using the disclosed methods can be derived from, for example without limitation, a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells.

In some embodiments, an engineered immune cell or a population of immune cells that are generated using the disclosed methods is obtained or prepared from peripheral blood. In some embodiments, an engineered immune cell is obtained or prepared from peripheral blood mononuclear cells (PBMCs). In some embodiments, an engineered immune cell is obtained or prepared from bone marrow. In some embodiments, an engineered immune cell is obtained or prepared from umbilical cord blood. In some embodiments, the cell is a human cell. In some embodiments, the cell is transfected or transduced by the nucleic acid vector using a method selected from the group consisting of electroporation, sonoporation, biolistics (e.g., Gene Gun), lipid transfection, polymer transfection, nanoparticles, viral transfection (e.g., retrovirus, lentivirus, AAV) or polyplexes.

In some embodiments, the engineered immune cells expressing at their cell surface membrane an antigen-specific CAR comprise a percentage of stem cell memory and central memory cells greater than 10%, 20%, 30%, 40%, 50%, 60%, 70%, 80%, 90%, or 100%.

In some embodiments, engineered immune cells expressing at their cell surface membrane an antigen-specific CAR comprise a percentage of stem cell memory and central memory cells of about 10% to about 60%, about 10% to about 50%, about 10% to about 40%, about 15% to about 50%, about 15% to about 40%, about 20% to about 60%, or about 20% to about 70%.

In some embodiments, engineered immune cells expressing at their cell surface membrane an antigen-specific CAR enriched in T_{CM} and/or T_{SCM} cells such that the engineered immune cells comprise at least about 60%, 65%, 70%, 75%, or 80% combined T_{CM} and T_{SCM} cells. In some embodiments, engineered immune cells expressing at their cell surface membrane an antigen-specific CAR are enriched in T_{CM} and/or T_{SCM} cells such that the engineered immune cells comprise at least about 70% combined T_{CM} and T_{SCM} cells. In some embodiments, engineered immune cells expressing at their cell surface membrane an antigen-specific CAR e enriched in T_{CM} and/or T_{SCM} cells such that the engineered immune cells comprise at least about 75% combined T_{CM} and/or T_{SCM} cells.

In some embodiments, an engineered immune cell is an inflammatory T-lymphocyte that expresses a CAR. In some embodiments, an engineered immune cell is a cytotoxic T-lymphocyte that expresses a CAR. In some embodiments, an engineered immune cell is a regulatory T-lymphocyte that expresses a CAR. In some embodiments, an engineered immune cell is a helper T-lymphocyte that expresses a CAR.

### Genetic Modification of CAR T Cells

In some embodiments, an engineered immune cell according to the present disclosure can comprise one or more disrupted or inactivated genes. In some embodiments, a gene for a target antigen (e.g., EGFRvIII, Flt3, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, BCMA, FLT3, CD70, DLL3, or CD34, CD70) can be knocked out to introduce a CAR targeting the same antigen (e.g., a EGFRvIII, Flt3, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, BCMA, FLT3, CD70, DLL3, or CD34, CD70 CAR) to avoid induced CAR activation. As described herein, in some embodiments, an engineered immune cell according to the present disclosure comprises one disrupted or inactivated gene selected from the group consisting of MHC1 (β2M), MHC2 (CIITA), EGFRvIII, Flt3, WT-1, CD20, CD23, CD30, CD38, CD33, CD133, MHC-WT1, TSPAN10, MHC-PRAME, Liv1, ADAM10, CHRNA2, LeY, NKGD2D, CS1, CD44v6, ROR1, Claudin-18.2, Muc17, FAP alpha, Ly6G6D, c6orf23, G6D, MEGT1, NG25, CD19, BCMA, FLT3, CD70, DLL3, or CD34, CD70, TCRα and TCRβ and/or expresses a CAR or a multi-chain CAR. In some embodiments, a cell comprises a multi-chain CAR. In some embodiments, the isolated cell comprises two disrupted or inactivated genes selected from the group consisting of: CD52 and TCRα, CDR52 and TCRβ, PD-1 and TCRα, PD-1 and TCRβ, MHC-1 and TCRα, MHC-1 and TCRβ, MHC2 and TCRα, MHC2 and TCRβ and/or expresses a CAR or a multi-chain CAR.

In some embodiments, an engineered immune cell according to the present disclosure comprises one disrupted or inactivated gene selected from the group consisting of CD52, DLL3, GR, PD-1, CTLA-4, LAG3, TIM3, BTLA, BY55, TIGIT, B7H5, LAIR1, SIGLEC10, 2B4, HLA, TCRα and TCRβ and/or expresses a CAR, a multi-chain CAR and/or a pTα transgene. In some embodiments, an isolated cell comprises polynucleotides encoding polypeptides comprising a multi-chain CAR. In some embodiments, the isolated cell according to the present disclosure comprises two disrupted or inactivated genes selected from the group consisting of: CD52 and GR, CD52 and TCRα, CDR52 and TCRβ, DLL3 and CD52, DLL3 and TCRα, DLL3 and TCRβ, GR and TCRα, GR and TCRβ, TCRα and TCRβ, PD-1 and TCRα, PD-1 and TCRβ, CTLA-4 and TCRα, CTLA-4 and TCRβ, LAG3 and TCRα, LAG3 and TCRβ, TIM3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ and/or expresses a CAR, including a multi-chain CAR, and/or a pTα transgene. In some embodiments the method comprises disrupting or inactivating one or more genes by introducing into the cells an endonuclease capable of selectively inactivating a gene by selective DNA cleavage. In some embodiments the endonuclease can be, for example, a zinc finger nuclease (ZFN), megaTAL nuclease, meganuclease, transcription activator-like effector nuclease (TALE-nuclease, or TALEN®), or CRISPR (e.g., Cas9 or Cas12) endonuclease.

In some embodiments, TCR is rendered not functional in the cells according to the disclosure by disrupting or inactivating TCRα gene and/or TCRβ gene(s). In some embodiments, a method to obtain modified cells derived from an individual is provided, wherein the cells can proliferate independently of the major histocompatibility complex (MHC) signaling pathway. Modified cells, which can proliferate independently of the MHC signaling pathway, susceptible to be obtained by this method are encompassed in the scope of the present disclosure. Modified cells disclosed herein can be used in for treating patients in need thereof against Host versus Graft (HvG) rejection and Graft versus Host Disease (GvHD); therefore in the scope of the present disclosure is a method of treating patients in need thereof against Host versus Graft (HvG) rejection and Graft versus Host Disease (GvHD) comprising treating said patient by administering to said patient an effective amount of modified cells comprising disrupted or inactivated TCRα and/or TCRβ genes.

The present disclosure provides methods of determining the purity of a population of engineered immune cells lacking or having reduced endogenous TCR expression. In some embodiments, the engineered immune cells comprise less than 5.0%, less than 4.0%, less than 3.0% TCR+ cells, less than 2.0% TCR+ cells, less than 1.0% TCR+ cells, less than 0.9% TCR+ cells, less than 0.8% TCR+ cells, less than 0.7% TCR+ cells, less than 0.6% TCR+ cells, less than 0.5% TCR+ cells, less than 0.4% TCR+ cells, less than 0.3% TCR+ cells, less than 0.2% TCR+ cells, or less than 0.1% TCR+ cells. Such a population can be a product of the disclosed methods.

In some embodiments, the immune cells are engineered to be resistant to one or more chemotherapy drugs. The chemotherapy drug can be, for example, a purine nucleotide analogue (PNA), thus making the immune cell suitable for cancer treatment combining adoptive immunotherapy and chemotherapy. Exemplary PNAs include, for example, clofarabine, fludarabine, cyclophosphamide, and cytarabine, alone or in combination. PNAs are metabolized by deoxycytidine kinase (dCK) into mono-, di-, and tri-phosphate PNA. Their tri-phosphate forms compete with ATP for DNA synthesis, act as pro-apoptotic agents, and are potent inhibitors of ribonucleotide reductase (RNR), which is involved in trinucleotide production.

In some embodiments, isolated cells or cell lines of the disclosure can comprise a pTα or a functional variant thereof. In some embodiments, an isolated cell or cell line can be further genetically modified by disrupting or inactivating the TCRα gene.

The disclosure also provides engineered immune cells comprising any of the CAR polynucleotides described herein. In some embodiments, a CAR can be introduced into an immune cell as a transgene via a plasmid vector. In some embodiments, the plasmid vector can also contain, for example, a selection marker which provides for identification and/or selection of cells which received the vector.

CAR polypeptides can be synthesized in situ in the cell after introduction of polynucleotides encoding the CAR polypeptides into the cell. Alternatively, CAR polypeptides can be produced outside of cells, and then introduced into cells. Methods for introducing a polynucleotide construct into cells are known in the art. In some embodiments, stable transformation methods (e.g., using a lentiviral vector) can be used to integrate the polynucleotide construct into the genome of the cell. In other embodiments, transient transformation methods can be used to transiently express the polynucleotide construct, and the polynucleotide construct not integrated into the genome of the cell. In other embodiments, virus-mediated methods can be used. The polynucleotides can be introduced into a cell by any suitable means such as for example, recombinant viral vectors (e.g., retroviruses, adenoviruses), liposomes, and the like. Transient transformation methods include, for example without limitation, microinjection, electroporation or particle bombardment. Polynucleotides can be included in vectors, such as for example plasmid vectors or viral vectors.

In some embodiments, isolated nucleic acids are provided comprising a promoter operably linked to a first polynucleotide encoding an antigen binding domain, at least one costimulatory molecule, and an activating domain. In some embodiments, the nucleic acid construct is contained within a viral vector. In some embodiments, the viral vector is selected from the group consisting of retroviral vectors, murine leukemia virus vectors, SFG vectors, adenoviral vectors, lentiviral vectors, adeno-associated virus (AAV) vectors, Herpes virus vectors, and vaccinia virus vectors. In some embodiments, the nucleic acid is contained within a plasmid.

In some embodiments, the isolated nucleic construct is contained within a viral vector and is introduced into the genome of an engineered immune cell by random integration, e.g., lentiviral- or retroviral-mediated random integration. In some embodiments, the isolated nucleic acid construct is contained in a viral vector or a non-viral vector and is introduced into the genome of an engineered immune cell by site-specific integration, e.g., adenovirus-mediated site-specific integration.

### 3. Manufacture of Engineered Immune Cells (Including CAR T Cells)

Provided herein are methods of analyzing or determining various attributes of engineered immune cells from a population of immune cells (including engineered immune cells such as CAR expressing or CAR+ cells). As described herein, engineered immune cells, such as CAR T cells, can be modified to reduce or eliminate expression of endogenous TCR, and the remaining TCR+ engineered immune cells can be depleted according to the methods described herein, at the end of production. The instant disclosure provides methods of characterizing or analyzing a population of engineered immune cells to characterize the drug product or as part of the manufacturing process. The instant disclosure also provides methods of analyzing or determining other attributes, such as the potency or polyfunctionality of the engineered immune cells to characterize the drug product or as part of the manufacturing process. In some embodiments, the engineered immune cells, such as CAR T cells, are manufactured according to Good Manufacturing Practice (GMP).

A variety of known techniques can be utilized in making the polynucleotides, polypeptides, vectors, antigen binding domains, immune cells, compositions, and the like according to the disclosure.

Prior to the in vitro manipulation or genetic modification of the immune cells described herein, the cells can be obtained from a subject. Cells expressing a CAR can be derived from an allogeneic or autologous source and can be depleted of endogenous TCR as described herein.

a. Source Material

In some embodiments, the immune cells comprise T cells. T cells can be obtained from a number of sources, including peripheral blood mononuclear cells (PBMCs), bone marrow, lymph nodes tissue, cord blood, thymus tissue, tissue from a site of infection, ascites, pleural effusion, spleen tissue, and tumors. In certain some embodiments, T cells can be obtained from a volume of blood collected from the subject using any number of techniques known to the skilled person, such as FICOLL™ separation.

Cells can be obtained from the circulating blood of an individual by apheresis. The apheresis product typically contains lymphocytes, including T cells, monocytes, granulocytes, B cells, other nucleated white blood cells, red blood cells, and platelets. In certain some embodiments, the cells collected by apheresis can be washed to remove the plasma fraction, and then placed in an appropriate buffer or media for subsequent processing.

In certain some embodiments, T cells are isolated from PBMCs by lysing the red blood cells and depleting the monocytes, for example, using centrifugation through a PERCOLL™ gradient. A specific subpopulation of T cells, (e.g., CD28+, CD4+, CD45RA−, and CD45RO+ T cells or CD28+, CD4+, CDS+, CD45RA−, CD45RO+, and CD62L+ T cells) can be further isolated by positive or negative selection techniques known in the art. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. Flow cytometry and cell sorting can also be used to isolate cell populations of interest for use in the present disclosure.

PBMCs can be used directly for genetic modification with the immune cells (such as CARs or TCRs) using methods as described herein. In certain embodiments, after isolating the PBMCs, T lymphocytes can be further isolated and both cytotoxic and helper T lymphocytes can be sorted into naive, memory, and effector T cell subpopulations either before or after genetic modification and/or expansion. In some embodiments, CD8+ cells are further sorted into naive, stem cell memory, central memory, and effector cells by identifying cell surface antigens that are associated with each of these types of CD8+ cells. In some embodiments, the expression of phenotypic markers of central memory T cells include CD27, CD45RA, CD45RO, CD62L, CCR7, CD28, CD3, and CD127 and are negative for granzyme B. In some embodiments, stem cell memory T cells are CD45RO−, CD62L+, CD8+ T cells. In some embodiments, central memory T cells are CD45RO+, CD62L+, CD8+ T cells. In some embodiments, effector T cells are negative for CD62L, CCR7, CD28, and CD127, and positive for granzyme B and perforin. In certain some embodiments, CD4+ T cells are further sorted into subpopulations. For example, CD4+ T helper cells can be sorted into naive, central memory, and effector cells by identifying cell populations that have cell surface antigens.

b. Stem Cell Derived Immune Cells

In some embodiments, the immune cells can be derived from embryonic stem (ES) or induced pluripotent stem (iPS) cells. Suitable HSCs, mesenchymal, iPS cells and other types of stem cells can be cultivated immortal cell lines or isolated directly from a patient. Various methods for isolating, developing, and/or cultivating stem cells are known in the art and can be used to practice the present disclosure.

In some embodiments, the immune cell is an induced pluripotent stem cell (iPSC) derived from a reprogrammed T-cell. In some embodiments, the source material can be an induced pluripotent stem cell (iPSC) derived from a T cell or a non-T cell. The source material can be an embryonic stem cell. The source material can be a B cell, or any other cell from peripheral blood mononuclear cell isolates, hematopoietic progenitor, hematopoietic stem cell, mesenchymal stem cell, adipose stem cell, or any other somatic cell type.

c. Genetic Modification of Isolated Cells

The immune cells, such as T cells, can be genetically modified following isolation using known methods, or the immune cells can be activated and expanded (or differentiated in the case of progenitors) in vitro prior to being genetically modified. In some embodiments, the isolated immune cells are genetically modified to reduce or eliminate expression of endogenous TCRα and/or CD52. In some embodiments, the cells are genetically modified using gene editing technology (e.g., CRISPR/Cas9, CRISPR/Cas12a, a zinc finger nuclease (ZFN), a TALEN, a MegaTAL, a meganuclease) to reduce or eliminate expression of endogenous proteins (e.g., TCRα and/or CD52). In another embodiment, the immune cells, such as T cells, are genetically modified with the chimeric antigen receptors described herein (e.g., transduced with a viral vector comprising one or more nucleotide sequences encoding a CAR) and then are activated and/or expanded in vitro.

Certain methods for making the constructs and engineered immune cells of the disclosure are described in PCT application PCT/US15/14520, the contents of which are hereby incorporated by reference in their entirety.

It will be appreciated that PBMCs can further include other cytotoxic lymphocytes such as NK cells or NKT cells. An expression vector carrying the coding sequence of a chimeric receptor as disclosed herein can be introduced into a population of human donor T cells, NK cells or NKT cells. Successfully transduced T cells that carry the expression vector can be sorted using flow cytometry to isolate CD3 positive T cells and then further propagated to increase the number of these CAR expressing T cells in addition to cell activation using anti-CD3 antibodies and IL-2 or other methods known in the art as described elsewhere herein. Standard procedures are used for cryopreservation of T cells expressing the CAR for storage and/or preparation for use in a human subject. In one embodiment, the in vitro transduction, culture and/or expansion of T cells are performed in the absence of non-human animal derived products such as fetal calf serum and fetal bovine serum.

For cloning of polynucleotides, the vector can be introduced into a host cell (an isolated host cell) to allow replication of the vector itself and thereby amplify the copies of the polynucleotide contained therein. The cloning vectors can contain sequence components generally include, without limitation, an origin of replication, promoter sequences, transcription initiation sequences, enhancer sequences, and selectable markers. These elements can be selected as appropriate by a person of ordinary skill in the art. For example, the origin of replication can be selected to promote autonomous replication of the vector in the host cell.

In certain some embodiments, the present disclosure provides isolated host cells containing the vector provided herein. The host cells containing the vector can be useful in expression or cloning of the polynucleotide contained in the vector. Suitable host cells can include, without limitation, prokaryotic cells, fungal cells, yeast cells, or higher eukaryotic cells such as mammalian cells, particularly human cells.

The vector can be introduced to the host cell using any suitable methods known in the art, including, without limitation, DEAE-dextran mediated delivery, calcium phosphate precipitate method, cationic lipids mediated delivery, liposome mediated transfection, electroporation, microprojectile bombardment, receptor-mediated gene delivery, delivery mediated by polylysine, histone, chitosan, and peptides. Standard methods for transfection and transformation of cells for expression of a vector of interest are well known in the art. In a further embodiment, a mixture of different expression vectors can be used in genetically modifying a donor population of immune effector cells wherein each vector encodes a different CAR as disclosed herein. The resulting transduced immune effector cells form a mixed population of engineered cells, with a proportion of the engineered cells expressing more than one different CARs.

In one embodiment, the disclosure provides a method of storing genetically engineered cells expressing CARs or TCRs. This involves cryopreserving the immune cells such that the cells remain viable upon thawing. A fraction of the immune cells expressing the CARs can be cryopreserved by methods known in the art to provide a permanent source of such cells for the future treatment of patients afflicted with a malignancy. When needed, the cryopreserved transformed immune cells can be thawed, grown and expanded for more such cells.

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion media can be any isotonic medium formulation, typically normal saline, Normosol™ R (Abbott) or Plasma-Lyte™ A (Baxter), but also 5% dextrose in water or Ringer's lactate can be utilized. The infusion medium can be supplemented with human serum albumin.

d. Allogeneic CAR T Cells

The process for manufacturing allogeneic CAR T therapy involves harvesting healthy, selected, screened and tested T cells from healthy donors. Next, the T cells are engineered to express CARs, which recognize certain cell surface proteins that are expressed in hematologic or solid tumors. Allogeneic T cells are gene editing to reduce the risk of graft versus host disease (GvHD) and to prevent allogeneic rejection. A T cell receptor gene (e.g., TCRα, TCRβ) is knocked out to avoid GvHD. The CD52 gene can be knocked out to render the CAR T product resistant to anti-CD52 antibody treatment. Anti-CD52 antibody treatment can therefore be used to suppress the host immune system and allow the CAR T to stay engrafted to achieve full therapeutic impact. The engineered T cells then undergo a purification step and are ultimately cryopreserved in vials for delivery to patients.

e. Autologous CAR T Cells

Autologous chimeric antigen receptor (CAR) T cell therapy, involves collecting a patient's own cells (e.g., white blood cells, including T cells) and genetically engineering the T cells to express CARs that recognize target expressed on the cell surface of one or more specific cancer cells and kill cancer cells. The engineered cells are then cryopreserved and subsequently administered to the patient.

### 4. Methods of In Vitro Sorting

In some embodiments, provided are methods for in vitro sorting of a population of immune cells, wherein a subset of the population of immune cells comprises engineered immune cells expressing an antigen-specific CARs comprising epitopes specific for monoclonal antibodies (e.g., exemplary mimotope sequences). The method comprises contacting the population of immune cells with a monoclonal antibody specific for the epitopes and selecting the immune cells that bind to the monoclonal antibody to obtain a population of cells enriched in engineered immune cells expressing an antigen-specific CAR.

In some embodiments, said monoclonal antibody specific for said epitope is optionally conjugated to a fluorophore. In this embodiment, the step of selecting the cells that bind to the monoclonal antibody can be done by Fluorescence Activated Cell Sorting (FACS).

In some embodiments, said monoclonal antibody specific for said epitope is optionally conjugated to a magnetic particle. In this embodiment, the step of selecting the cells that bind to the monoclonal antibody can be done by Magnetic Activated Cell Sorting (MACS).

In some embodiments, the mAb used in the method for sorting immune cells expressing the CAR is chosen from alemtuzumab, ibritumomab tiuxetan, muromonab-CD3, tositumomab, abciximab, basiliximab, brentuximab vedotin, cetuximab, infliximab, rituximab, bevacizumab, certolizumab pegol, daclizumab, eculizumab, efalizumab, gemtuzumab, natalizumab, omalizumab, palivizumab, ranibizumab, tocilizumab, trastuzumab, vedolizumab, adalimumab, belimumab, canakinumab, denosumab, golimumab, ipilimumab, ofatumumab, panitumumab, QBEND-10 and/or ustekinumab. In some embodiments, said mAb is rituximab. In another embodiment, said mAb is QBEND-10. In other embodiments the mAb binds to TCRα, or TCRβ.

In some embodiments, the population CAR-expressing immune cells obtained when using the method for in vitro sorting CAR-expressing immune cells described above, comprises at least 70%, 75%, 80%, 85%, 90%, 95% of CAR-expressing immune cells. In some embodiments, the population of CAR-expressing immune cells obtained when using the method for in vitro sorting CAR-expressing immune cells, comprises at least 85% CAR-expressing immune cells.

In some embodiments, the population of CAR-expressing immune cells obtained when using the method for in vitro sorting CAR-expressing immune cells described above shows increased cytotoxic activity in vitro compared with the initial (non-sorted) cell population. In some embodiments, said cytotoxic activity in vitro is increased by 10%, 20%, 30%, 40% or 50%. In some embodiments, the immune cells are T-cells.

In some embodiments, the mAbs are previously bound onto a support or surface. Non-limiting examples of solid support can include a bead, agarose bead, a magnetic bead, a plastic welled plate, a glass welled plate, a ceramic welled plate, a column, or a cell culture bag.

The CAR-expressing immune cells to be administered to the recipient can be enriched in vitro from the source population. Methods of expanding source populations can include selecting cells that express an antigen such as CD34 antigen, using combinations of density centrifugation, immuno-magnetic bead purification, affinity chromatography, and fluorescent activated cell sorting.

Flow cytometry can be used to quantify specific cell types within a population of cells. In general, flow cytometry is a method for quantifying components or structural features of cells primarily by optical means. Since different cell types can be distinguished by quantifying structural features, flow cytometry and cell sorting can be used to count and sort cells of different phenotypes in a mixture.

In some embodiments, the method used for sorting T cells expressing CAR is the Magnetic-Activated Cell Sorting (MACS). Magnetic-activated cell sorting (MACS) is a method for separation of various cell populations depending on their surface antigens (e.g., CD molecules) by using superparamagnetic nanoparticles and columns. MACS can be used to obtain a pure cell population. Cells in a single-cell suspension can be magnetically labeled with microbeads. The sample is applied to a column composed of ferromagnetic spheres, which are covered with a cell-friendly coating allowing fast and gentle separation of cells. The unlabeled cells pass through while the magnetically labeled cells are retained within the column. The flow-through can be collected as the unlabeled cell fraction. After a washing step, the column is removed from the separator, and the magnetically labeled cells are eluted from the column.

Detailed protocol for the purification of specific cell population such as T-cell can be found in Basu S et al. (2010). (Basu S, Campbell H M, Dittel B N, Ray A. Purification of specific cell population by fluorescence activated cell sorting (FACS). J Vis Exp. (41): 1546).

### 5. Pharmaceutical Compositions and Therapy

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion media can be any isotonic medium formulation, typically normal saline, Normosol™ R (Abbott) or Plasma-Lyte™ A (Baxter), but also 5% dextrose in water or Ringer's lactate can be utilized. The infusion medium can be supplemented with human serum albumin.

In embodiments, desired treatment amounts of cells in the composition are generally at least 2 cells (for example, at least 1 CD8+ central or stem cell memory T cell and at least 1 CD4+ helper T cell subset; or two or more CD8+ central or stem cell memory T cell; or two or more CD4+ helper T cell subset) or is more typically greater than 10² cells, and up to and including 10⁶, up to and including 10⁷, 10⁸ or 10⁹ cells and can be more than 10¹⁰ cells. The number of cells will depend upon the desired use for which the composition is intended, and the type of cells included therein. The density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. In some aspects of the present disclosure, particularly since all the infused cells will be redirected to a particular target antigen, lower numbers of cells, in the range of about 10⁵/kilogram or about 10⁶/kilogram (10⁶-10¹¹ per patient) can be administered. CAR treatments can be administered multiple times at dosages within these ranges. The cells can be autologous, allogeneic, or heterologous to the patient undergoing therapy.

The CAR expressing cell populations of the present disclosure can be administered either alone, or as a pharmaceutical composition in combination with diluents and/or with other components such as IL-2 or other cytokines or cell populations. Pharmaceutical compositions of the present disclosure can comprise a CAR or TCR expressing cell population, such as T cells, as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are preferably formulated for intravenous administration.

The pharmaceutical compositions (solutions, suspensions or the like), can include one or more of the following: sterile diluents such as water for injection, saline solution, preferably physiological saline, Ringer's solution, isotonic sodium chloride, fixed oils such as synthetic mono- or diglycerides which can serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampules, disposable syringes or multiple dose vials made of glass or plastic. An injectable pharmaceutical composition is preferably sterile.

### 6. Methods of Treatment

The disclosure comprises methods for treating or preventing a disease (e.g., cancer) in a patient, comprising administering to a patient in need thereof an effective amount of CAR T cells, or engineered immune cells comprising a CAR disclosed herein. In some embodiments, the effective amount of CAR T cells or engineered immune cells have been analyzed for various attributes according to the methods described in the instant disclosure. In some embodiments, the CAR T cell drug product for therapeutic use has been analyzed for various attributes, such as potency or polyfunctionality according to the methods described in the instant disclosure. In some embodiments, the CAR T cells are TCR− CAR T cells, and the CAR T drug product for therapeutic use has been analyzed for various attributes, such as the amount or percentage of remaining TCR+ CAR T cells and/or potency or polyfunctionality according to the methods described in the instant disclosure.

Methods are provided for treating diseases or disorders, including cancer. In some embodiments, the disclosure relates to creating a T cell-mediated immune response in a subject, comprising administering an effective amount of the engineered immune cells of the present application to the subject. In some embodiments, the T cell-mediated immune response is directed against a target cell or cells. In some embodiments, the engineered immune cell comprises a chimeric antigen receptor (CAR). In some embodiments, the target cell is a tumor cell. In some aspects, the disclosure comprises a method for treating or preventing a malignancy, said method comprising administering to a subject in need thereof an effective amount of at least one isolated antigen binding domain described herein. In some aspects, the disclosure comprises a method for treating or preventing a malignancy, said method comprising administering to a subject in need thereof an effective amount of at least one immune cell, wherein the immune cell comprises at least one chimeric antigen receptor, T cell receptor, and/or isolated antigen binding domain as described herein. The CAR containing immune cells of the disclosure can be used to treat malignancies involving aberrant expression of biomarkers. In some embodiments, CAR containing immune cells of the disclosure can be used to treat small cell lung cancer, melanoma, low grade gliomas, glioblastoma, medullary thyroid cancer, carcinoids, dispersed neuroendocrine tumors in the pancreas, bladder and prostate, testicular cancer, and lung adenocarcinomas with neuroendocrine features. In exemplary embodiments, the CAR containing immune cells, e.g., CAR-T cells of the disclosure are used to treat small cell lung cancer.

Also provided are methods for reducing the size of a tumor in a subject, comprising administering to the subject an engineered cell of the present disclosure to the subject, wherein the cell comprises a chimeric antigen receptor comprising an antigen binding domain and binds to an antigen on the tumor.

In some embodiments, the subject has a solid tumor, or a blood malignancy such as lymphoma or leukemia. In some embodiments, the engineered cell is delivered to a tumor bed. In some embodiments, the cancer is present in the bone marrow of the subject. In some embodiments, the engineered cells are autologous immune cells, e.g., autologous T cells. In some embodiments, the engineered cells are allogeneic immune cells, e.g., allogeneic T cells. In some embodiments, the engineered cells are heterologous immune cells, e.g., heterologous T cells. In some embodiments, the engineered cells of the present application are transfected or transduced in vivo. In other embodiments, the engineered cells are transfected or transduced ex vivo. As used herein, the term “in vitro cell” refers to any cell which is cultured ex vivo.

A “therapeutically effective amount,” “effective dose,” “effective amount,” or “therapeutically effective dosage” of a therapeutic agent, e.g., engineered CART cells, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The terms “patient” and “subject” are used interchangeably and include human and non-human animal subjects as well as those with formally diagnosed disorders, those without formally recognized disorders, those receiving medical attention, those at risk of developing the disorders, etc.

The term “treat” and “treatment” includes therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. The term “prevent” does not require the 100% elimination of the possibility of an event. Rather, it denotes that the likelihood of the occurrence of the event has been reduced in the presence of the compound or method.

Desired treatment amounts of cells in the composition is generally at least 2 cells (for example, at least 1 CD8+ central memory T cell and at least 1 CD4+ helper T cell subset) or is more typically greater than 10² cells, and up to 10⁶, up to and including 10⁸ or 10⁹ cells and can be more than 10¹⁰ cells. The number of cells will depend upon the desired use for which the composition is intended, and the type of cells included therein. The density of the desired cells is typically greater than 10⁶ cells/ml and generally is greater than 10⁷ cells/ml, generally 108 cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. In some aspects of the present disclosure, particularly since all the infused cells will be redirected to a particular target antigen, lower numbers of cells, in the range of 10⁶/kilogram (10⁶-10¹¹ per patient) can be administered. CAR treatments can be administered multiple times at dosages within these ranges. The cells can be autologous, allogeneic, or heterologous to the patient undergoing therapy.

In some embodiments, the therapeutically effective amount of the CAR T cells is about 1×10⁵ cells/kg, about 2×10⁵ cells/kg, about 3×10⁵ cells/kg, about 4×10⁵ cells/kg, about 5×10⁵ cells/kg, about 6×10⁵ cells/kg, about 7×10⁵ cells/kg, about 8×10⁵ cells/kg, about 9×10⁵ cells/kg, 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg.

In some embodiments, target doses for CAR+/CAR-T+/TCR+ cells range from 1×10⁶-2×10⁸ cells/kg, for example 2×10⁶ cells/kg. It will be appreciated that doses above and below this range can be appropriate for certain subjects, and appropriate dose levels can be determined by the healthcare provider as needed. Additionally, multiple doses of cells can be provided in accordance with the disclosure.

In some aspect, the disclosure comprises a pharmaceutical composition comprising at least one antigen binding domain as described herein and a pharmaceutically acceptable excipient. In some embodiments, the pharmaceutical composition further comprises an additional active agent.

The CAR expressing cell populations of the present disclosure can be administered either alone, or as a pharmaceutical composition in combination with diluents and/or with other components such as IL-2 or other cytokines or cell populations. Pharmaceutical compositions of the present disclosure can comprise a CAR or TCR expressing cell population, such as T cells, as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are preferably formulated for intravenous administration.

The pharmaceutical compositions (solutions, suspensions or the like), can include one or more of the following: sterile diluents such as water for injection, saline solution, preferably physiological saline, Ringer's solution, isotonic sodium chloride, fixed oils such as synthetic mono- or diglycerides which can serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampules, disposable syringes or multiple dose vials made of glass or plastic. An injectable pharmaceutical composition is preferably sterile.

In some embodiments, upon administration to a patient, engineered immune cells expressing at their cell surface any one of the antigen-specific CARs described herein can reduce, kill or lyse endogenous antigen-expressing cells of the patient. In one embodiment, a percentage reduction or lysis of antigen-expressing endogenous cells or cells of a cell line expressing an antigen by engineered immune cells expressing any one of an antigen-specific CARs described herein is at least about or greater than 10%, 15%, 20%, 25%, 30%, 35%, 40%, 45%, 50%, 55%, 60%, 65%, 70%, 75%, 80%, 85%, 90%, or 95%. In one embodiment, a percentage reduction or lysis of antigen-expressing endogenous cells or cells of a cell line expressing an antigen by engineered immune cells expressing antigen-specific CARs is about 5% to about 95%, about 10% to about 95%, about 10% to about 90%, about 10% to about 80%, about 10% to about 70%, about 10% to about 60%, about 10% to about 50%, about 10% to about 40%, about 20% to about 90%, about 20% to about 80%, about 20% to about 70%, about 20% to about 60%, about 20% to about 50%, about 25% to about 75%, or about 25% to about 60%. In one embodiment, the endogenous antigen-expressing cells are endogenous antigen-expressing bone marrow cells.

In one embodiment, the percent reduction or lysis of target cells, e.g., a cell line expressing an antigen, by engineered immune cells expressing at their cell surface membrane an antigen-specific CAR of the disclosure can be measured using the assay disclosed herein.

The methods can further comprise administering one or more chemotherapeutic agent. In certain some embodiments, the chemotherapeutic agent is a lymphodepleting (preconditioning) chemotherapeutic. For example, methods of conditioning a patient in need of a T cell therapy comprising administering to the patient specified beneficial doses of cyclophosphamide (between 200 mg/m²/day and 2000 mg/m²/day, about 100 mg/m²/day and about 2000 mg/m²/day; e.g., about 100 mg/m²/day, about 200 mg/m²/day, about 300 mg/m²/day, about 400 mg/m²/day, about 500 mg/m²/day, about 600 mg/m²/day, about 700 mg/m²/day, about 800 mg/m²/day, about 900 mg/m²/day, about 1000 mg/m²/day, about 1500 mg/m²/day or about 2000 mg/m²/day) and specified doses of fludarabine (between 20 mg/m²/day and 900 mg/m²/day, between about 10 mg/m²/day and about 900 mg/m²/day; e.g., about 10 mg/m²/day, about 20 mg/m²/day, about 30 mg/m²/day, about 40 mg/m²/day, about 40 mg/m²/day, about 50 mg/m²/day, about 60 mg/m²/day, about 70 mg/m²/day, about 80 mg/m²/day, about 90 mg/m²/day, about 100 mg/m²/day, about 500 mg/m²/day or about 900 mg/m²/day). A preferred dose regimen involves treating a patient comprising administering daily to the patient about 300 mg/m²/day of cyclophosphamide and about 30 mg/m²/day of fludarabine for three days prior to administration of a therapeutically effective amount of engineered T cells to the patient.

In some embodiments, lymphodepletion further comprises administration of a CD52 antibody. In some embodiments, the CD52 antibody is alemtuzumab. In some embodiments, the CD52 antibody is administered at a dose of about 13 mg/day IV.

In other embodiments, the antigen binding domain, transduced (or otherwise engineered) cells and the chemotherapeutic agent are administered each in an amount effective to treat the disease or condition in the subject.

In certain some embodiments, compositions comprising CAR-expressing immune effector cells disclosed herein can be administered in conjunction with any number of chemotherapeutic agents, which can be administered in any order. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′, 2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (TAXOL™, Bristol-Myers Squibb) and doxetaxel (TAXOTERE®, Rhone-Poulenc Rorer); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RF S2000; difluoromethylomithine (DMFO); retinoic acid derivatives such as Targretin™ (bexarotene), Panretin™, (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Combinations of chemotherapeutic agents are also administered where appropriate, including, but not limited to CHOP, i.e., Cyclophosphamide (Cytoxan®), Doxorubicin (hydroxydoxorubicin), Vincristine (Oncovin®), and Prednisone.

In some embodiments, the chemotherapeutic agent is administered at the same time or within one week after the administration of the engineered cell, polypeptide, or nucleic acid. In other embodiments, the chemotherapeutic agent is administered from 1 to 4 weeks or from 1 week to 1 month, 1 week to 2 months, 1 week to 3 months, 1 week to 6 months, 1 week to 9 months, or 1 week to 12 months after the administration of the engineered cell, polypeptide, or nucleic acid. In other embodiments, the chemotherapeutic agent is administered at least 1 month before administering the cell, polypeptide, or nucleic acid. In some embodiments, the methods further comprise administering two or more chemotherapeutic agents.

A variety of additional therapeutic agents can be used in conjunction with the compositions described herein. For example, potentially useful additional therapeutic agents include PD-1 inhibitors such as nivolumab (Opdivo®), pembrolizumab (Keytruda®), pembrolizumab, pidilizumab, and atezolizumab (Tcentriq®).

Additional therapeutic agents suitable for use in combination with the disclosure include, but are not limited to, ibrutinib (Imbruvica®), ofatumumab (Arzerra®, rituximab (Rituxan®), bevacizumab (Avastin®), trastuzumab (Herceptin®), trastuzumab emtansine (KADCYLA®, imatinib (Gleevec®), cetuximab (Erbitux®, panitumumab) (Vectibix®), catumaxomab, ibritumomab, ofatumumab, tositumomab, brentuximab, alemtuzumab, gemtuzumab, erlotinib, gefitinib, vandetanib, afatinib, lapatinib, neratinib, axitinib, masitinib, pazopanib, sunitinib, sorafenib, toceranib, lestaurtinib, axitinib, cediranib, lenvatinib, nintedanib, pazopanib, regorafenib, semaxanib, sorafenib, sunitinib, tivozanib, toceranib, vandetanib, entrectinib, cabozantinib, imatinib, dasatinib, nilotinib, ponatinib, radotinib, bosutinib, lestaurtinib, ruxolitinib, pacritinib, cobimetinib, selumetinib, trametinib, binimetinib, alectinib, ceritinib, crizotinib, aflibercept, adipotide, denileukin diftitox, mTOR inhibitors such as Everolimus and Temsirolimus, hedgehog inhibitors such as sonidegib and vismodegib, CDK inhibitors such as CDK inhibitor (palbociclib).

In some embodiments, the composition comprising CAR-containing immune cells can be administered with a therapeutic regimen to prevent cytokine release syndrome (CRS) or neurotoxicity. The therapeutic regimen to prevent cytokine release syndrome (CRS) or neurotoxicity can include lenzilumab, tocilizumab, atrial natriuretic peptide (ANP), anakinra, iNOS inhibitors (e.g., L-NIL or 1400W). In additional embodiments, the composition comprising CAR-containing immune cells can be administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Exemplary NSAIDs include ibuprofen, naproxen, naproxen sodium, Cox-2 inhibitors, and sialylates. Exemplary analgesics include acetaminophen, oxycodone, tramadol of proporxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline.

In certain embodiments, the compositions described herein are administered in conjunction with a cytokine. Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, IL-21 a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

### 7. Kits and Articles of Manufacture

The present disclosure provides kits comprising reagents for analyzing CAR T drug product according to the methods described herein. In some embodiments, the kit comprises one or more reagents for the detection of CD3 and TCRγδ, for example, an anti-CD3 antibody and an anti-TCRγδ antibody. In some embodiments, the kit further comprises one or more reagents for the detection of CD45, CD5, CD52, CD107a and/or a CAR. In some embodiments, the kit further comprises one or more reagents for the detection of TCRαβ. In some embodiments, the kit comprises one or more reagent for analyzing CAR T drug product according to the methods described herein, wherein the one or more reagent is conjugated with a detection label.

The present disclosure also provides kits comprising any of the cultured immune cells or engineered immune cells described herein, and pharmaceutical compositions of the same. In some exemplary embodiments, a kit of the disclosure comprises allogeneic CAR T cells for administering to a subject.

The present application further provides articles of manufacture comprising any one of the therapeutic compositions or kits described herein. Examples of an article of manufacture include vials (e.g. sealed vials).

The following examples are offered for illustrative purposes only. Indeed, various modifications of the invention in addition to those shown and described herein will become apparent to those skilled in the art from the foregoing description.

### EXAMPLES

### Allogeneic CAR T Cell Generation

CAR T cells were manufactured from healthy donor leukapheresate in a process involving the transduction of lentivirus harboring the CAR scFv transgene recognizing CD19. leukapheresate from an apheresis procedure was washed to remove platelets and Ficoll-purified to remove RBCs using the Sepax cell separation system. Cells were then seeded and activated with TransAct™ activation agent (MACS GMP T cell TransAct™) to induce activation and proliferation of the T cells. Cells were then plated in six-well plates with the LVV containing the construct that expresses the CD19(4G7) scFv/4-1BB/CD3ζ CAR. Then, TALEN© mRNAs were transfected into T cells by electroporation (EP) to genetically disrupt TRAC and CD52 genes and the TCRαβ and CD52 protein expression using the AgilePulse™ MTX system. Following expansion, CAR T cells were harvested and subjected to TCRαβ+ cell depletion to remove residual TCRαβ+ cells to a minimum level. The CliniMACS Prodigy, capable of automated and closed processing, was chosen for TCRαβ+ cell depletion. CAR T cells harvested from the bioreactor are subjected to cell concentration, antibody labelling and depletion on the Prodigy using the Miltenyi CliniMACS TCR α/β Reagent kit (anti-TCRαβ antibody clone BW242/412). The CD19− specific CAR T cells Allo-501 and Allo-501A were tested. Both types of CAR T cells expressing the same anti-CD19 scFv (based on clone 4G7); Allo-501 CAR T cells additionally express rituximab mimotope on the cell surface that are absent in Allo-501A CAR T cells.

### Cell Culture

Daudi cells (CD19 positive target tumor cells) and KG1a cells (CD19 negative control cells) were obtained from the American Type Culture Collection (VA, USA) and were maintained in complete medium comprised of Roswell Park Memorial Institute (RPMI) medium or Dulbecco's Modified Eagle Medium (DMEM) (Gibco®, Life Technologies, NY, USA) with 10% heat-inactivated fetal bovine serum (Gibco) and additives (2 mM glutamine, Gibco), 0.1 mM nonessential amino acids (Gibco), 100 μg/ml streptomycin (Sigma-Aldrich, MO, USA) and Gentamicin (10 ug/mL) (Sigma-Aldrich) in a humidified incubator at 37° C. with 5% CO2.

### Flow Cytometry

CD19 CAR expression was assessed using an anti-idiotype antibody recognizing the scFv Fab by intracellular staining. The anti-id antibody can be, for example, as described in WO2020/214937. To investigate the multifunctional capacity of CAR T cells upon target stimulation, intracellular cytokine staining was performed. After co-culture of Daudi cells (CD19 positive) or KG1a cells (CD19 negative), and co-incubation with fluorescently-labeled anti-human CD107a antibody, allogeneic CD19 CAR T cells were stained using a 15 color flow cytometry panel for immune cell lineages present in PBMCs (Table 1). CD19 allogeneic CAR T cells were also incubated under a pan-T cell stimulation condition, e.g., incubations in the presence of PMA (Phorbol Myristate Acetate) and Ionomycin (a calcium ionophore) without target cells as a positive control. During co-culture, antihuman CD107a-BV421, Monensin A (a protein transport inhibitor, BD Biosciences), and GolgiPlug™ (Pharmingen), was added to coculture wells and incubated together with target cells for 6 hrs or 18 hrs. Cells were then harvested and blocked with human IgG to remove non-specific binding, then stained with surface antibodies. After incubation and washing, cells were fixed and permeabilized with Cytofix/Cytoperm™ (Becton Dickinson) prior to blocking with human IgG (Sigma), then stained with intracellular cytokines and analyzed on the BD Fortessa X-20 using the FacsDIVA software. Viability of cells was determined by staining with Fixable far-red dye (Invitrogen, CA, USA). A lineage degranulation flow cytometry analysis panel is shown in Table 1, in which exemplary suitable detection reagents are shown. All antibodies, except the anti-TCRαβ (Miltenyi Biotech) and the anti-id (Allogene) antibodies, were obtained from Biolegend or BD Biosciences. Post-run .fcs files were compensated and manual gating performed using FlowJo from Treestar. Boolean gating on mutually exclusive cytokine positive populations (IFNγ, TNFα, and IL2) were performed to assess polyfunctionality within CD107a positive and negative populations, respectively.

TABLE 1

| | | | | | Exemplary | Exemplary |
|---|---|---|---|---|---|---|
| | Laser | Filter | Stage | Markers | Ab Clone | DYE |
| 1 | Violet | 450/50 | Culture | CD107a | H4A3 | BV421 |
| 2 | Red | 780/60 | Preincubation | Live/Dead | // | APC-Cy7 |
| | | | (serum-free | | | |
| | | | buffer) | | | |
| 3 | Red | 670/30 | Extracellular | TCRαβ | BW242/412 | APC |
| 4 | UV | 379/28 | | TCRγδ | 11F2 | BUV395 |
| 5 | Blue | 695/40 | | CD56 | NCAM16.2 | BB700 |
| 6 | Yellow | 780/60 | | CD45 | HI30 | PE-Cy7 |
| | Green | | | | | |
| 7 | Red | 730/45 | | CD4 | RPA-T4 | Alx700 |
| 8 | Violet | 525/50 | | CD8 | SK1 | V500 |
| 9 | Violet | 780/60 | | CD5 | UCTH2 | BV785 |
| 10 | Blue | 530/30 | | CD52 | HI186 | FITC |
| 11 | Violet | 710/50 | | CD3 | SK7 | BV711 |
| 12 | Yellow | 586/15 | Intracellular | CAR | A8 (raised | PE |
| | Green | | | (Anti-ID) | against 4G7) | |
| 13 | Violet | 610/20 | | IFNγ | B27 | BV605 |
| 14 | Violet | 670/30 | | IL-2 | MQ1-17H12 | BV650 |
| 15 | Yellow | 610/20 | | TNFα | MAb11 | PE-Dazzle594 |
| | Green | | | | | |

### Statistics

Samples were analyzed on Spotfire (Tibco) using the Pearson correlation coefficient analysis across at least the 5 attributes (CD107a, CD19 CAR, TNFα, IL2, and IFNγ), along with regression analyses.

### Example 1 TCRαβ+ Enumeration Based on an Internal Biological Control

Allogeneic CAR T cell products described above have the TRAC locus ablated by TALEN® gene editing to minimize the risks of GvHD (graft versus host disease) that may be caused by donor TCR recognizing host cell antigens as being foreign. After transduction of CAR and genetic modifications, the remaining TCRαβ+ donor CAR T cells were removed by anti-TCRαβ antibody-mediated depletion. After depletion, the amount of residual TCRαβ+ CAR T cells were typically directly measured by an immunoassay, e.g., flow cytometry. When possible, a compatible antibody that binds to a different epitope than the antibody used for depletion is chosen for detection. Reagent incompatibility may lead to inaccurate results. In addition, accurate direct measurement by flow cytometry relies on proper gate setting, i.e., setting a proper TCRαβ+ cutoff from donor-matching T cells. This experiment was designed to investigate whether the residual TCRαβ+ can be enumerated based on internal biological control instead of direct measurement.

Flow cytometry analyses were performed using a cocktail of detection reagents specific for T cell lineage markers: e.g., CD3, CD4, CD5, CD8, TCRαβ, and TCRγ6.

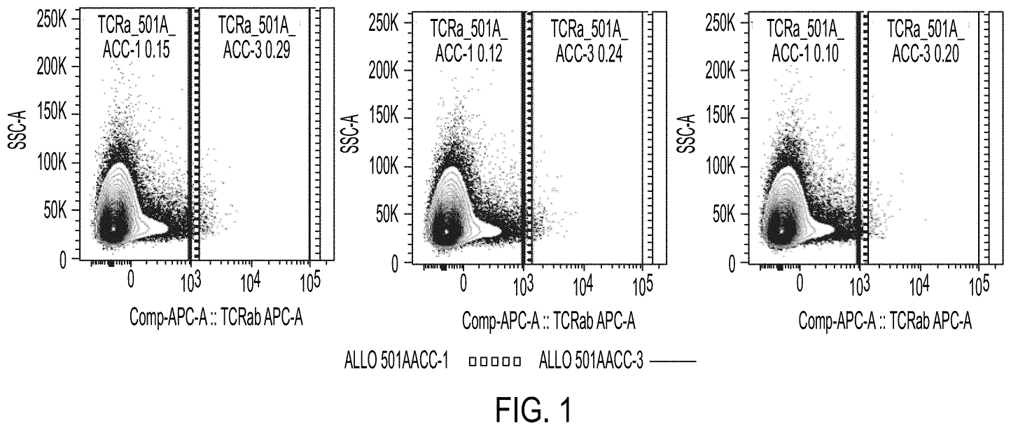

The residual TCRαβ+ cells were first analyzed by flow cytometry with an assigned gating. As shown in FIG. 1 and Table 2, different gating set based on different donors led to different readout. Thus, due to the lack of a reliable universal gating across all donor samples, accurate determination of the residual TCRαβ+ T cells in each batch of allogeneic CAR T cell manufacturing would have to rely on donor-specific gating controls. Donor-specific gating controls would require setting aside a sample of unmodified donor cells before the manufacturing process for determining the TCRαβ cutoff for the donor-matched drug product. This additional consideration further complicates the manufacturing process.

TABLE 2

| | R1 | R2 | R3 | Average | SD | % RSD |
|---|---|---|---|---|---|---|
| ALLO-501A TCRαβ + | 0.15 | 0.12 | 0.10 | 0.12 | 0.03 | 20% |
| gate set by Donor 1 | | | | | | |
| ALLO-501A TCRαβ + | 0.29 | 0.24 | 0.20 | 0.24 | 0.05 | 19% |
| gate set by Donor 2 | | | | | | |

To bypass the problem, the residual TCRαβ+ cells were next determined by using an internal biological control. CD3 staining is commonly used as an indication of T cells. Upon genetic ablation of TRAC, the protein complex of CD3 T cell co-receptor is destabilized at the cell surface and is no longer available for extra-cellular surface detection. We hypothesize that we can assess intracellular CD3 on TRAC-ablated T cells, and CD5 co-expressed with intracellular CD3 expression. We first tested whether CD5 can be utilized as another T cell antigen that is detectable and is not destabilized at the cell surface upon TRAC gene disruption.

Two flow panels were performed, one with intracellular CD3 staining and the other with surface staining, utilizing a backbone flow cytometry cocktail comprising T cell lineage markers: CD4, CD8, CD5. Flow cytometry analysis was performed by manually gating on T cell populations following gating logic applied in parallel samples to assess the total numbers of viable CD5+ events that were intracellular CD3+ and vice versa.

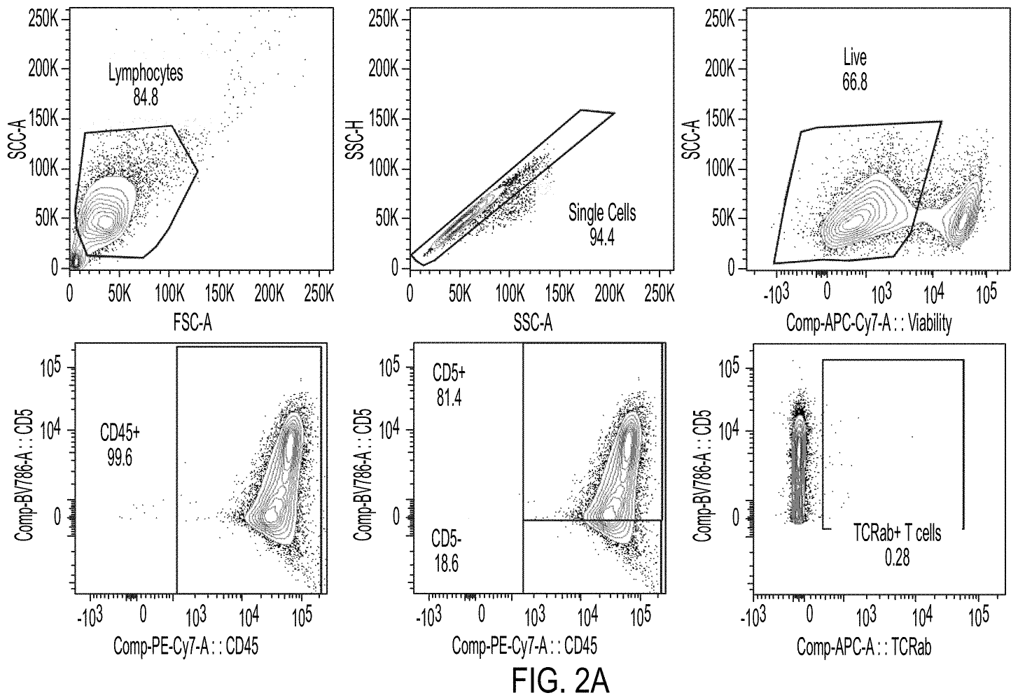

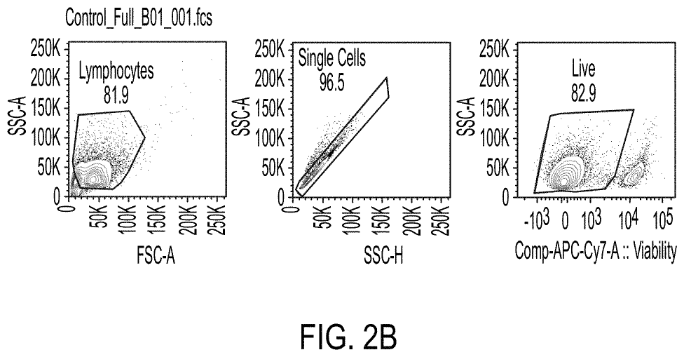

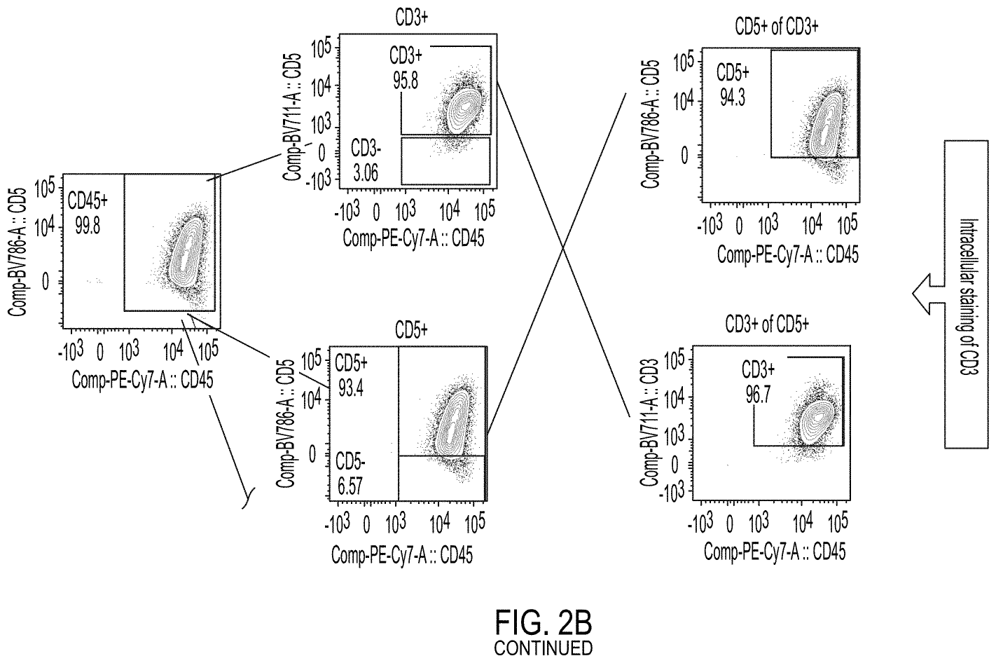

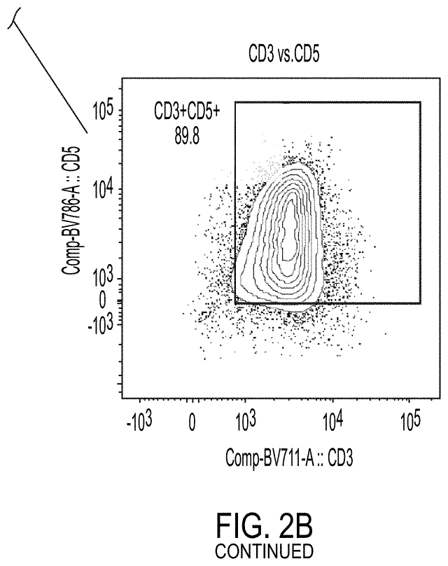

As shown in FIG. 2A, based on the gating of viable T cell populations, we confirmed the loss of extracellular CD3 upon TRAC-ablation in allogeneic CAR T cell product. We observed a correlation of extracellular CD5 with intracellular CD3 in TRAC-ablated allogeneic CAR T cell product, with populations that are 95.7% intracellular CD3+ showing up as 93.3% positive for surface CD5+. Additionally, 94.2% of intracellular CD3+ was surface CD5+ and 96.7% the converse. Lastly, 89.7% of all viable cells were double positive for surface CD5+ and intracellular CD3+. See FIG. 2B. The data is summarized in Table 3. Thus, it is feasible to use another surface expression marker CD5 as an indicator for T cells, for example, TRAC-ablated allogeneic CAR T cells.

TABLE 3

| ALLO-501A | | | % Intra | % surface | % intra |
|---|---|---|---|---|---|
| Gating: All | | % | CD3+ & | CD5+ of | CD3+ of |
| events/singlets/ | % intra | surface | surface | intra | surface |
| live/CD45+ | CD3+ | CD5+ | CD5+ | CD3+ | CD5+ |
| Full stain | 95.7 | 93.3 | 89.7 | 94.2 | 96.7 |

Next, FIG. 3 describes the processes of determining residual TCRαβ+ T cells after transduction of CAR and genetic ablation of TRAC, and in this case also the knockout of the CD52 gene. Panels A and B show the isolation of cells of interest based on size and granularity. Viable CD45+ T cells were isolated for further characterization (Panel C), and the surface expression of CD3 and CD5 were analyzed (Panel D). The cells were further analyzed for the expression of TCRαβ using an anti-TCRαβ detection antibody that was the same antibody used for depletion, i.e., clone BW242/412 (Panel E). The residual TCRαβ+ T cells was determined as 1.23% by direct measurement (Panel E). In parallel, the cells were analyzed for the expression of TCRγδ against CD3 extracellular staining. The results in Panel F show that the 92.2% of total cells are CD3- and TCRγδ− and the remaining 7.78% cells are surface CD3+ and TCRγδ+. Upon further analysis, the surface CD3+ cells from Panel D (7.48%) were all TCRγδ+(99.6% in Panel G), and the residual surface CD3+ cells amounted to 0.35% that represented the residual TCRαβ+ T cells.

The results indicate that the level of residual TCRαβ+ cells determined by direct detection (1.23%) was an over-estimation as compared to the determination by using an internal biological control (0.35%). A summary of analyses comparing the two methods and the error margin between the two methods from three samples from CAR T cells derived from two donors are shown in Table 4.

TABLE 4

| | | | % TCRαβ+ | % TCRαβ+ | |
|---|---|---|---|---|---|
| | | | masked by | determined by | |
| | | | TCRαβ | using an | |
| | % CD3+ of | | depletion Ab | internal | |
| | total | % TCRγδ- | clone | biological | |
| | CD45+ | of CD3+ | BW242/412 | control | % error |
| ALLO- | 4.9 | 7.3 | 0.4 | 0.2 | 48.1 |
| 501A | | | | | |
| Donor 1 | | | | | |
| ALLO- | 7.8 | 7.3 | 0.6 | 0.1 | 471.6 |
| 501 | | | | | |
| Donor 2 | | | | | |
| ALLO- | 8.7 | 7.6 | 0.7 | 0.2 | 229.5 |
| 501A | | | | | |
| Donor 2 | | | | | |

### Example 2 Increased Surface Expression of the Degranulation Marker CD107a Correlates with CAR T Cells with Enhanced Polyfunctionality

We next set out to investigate whether target-specific induction of CD107a correlates with the extent of effector cytokine induction across defined immune cell subsets. The experiment was designed to examine whether the induction of surface CD107 correlates with the increase of effector cytokines and enhanced killing by CAR T cells.

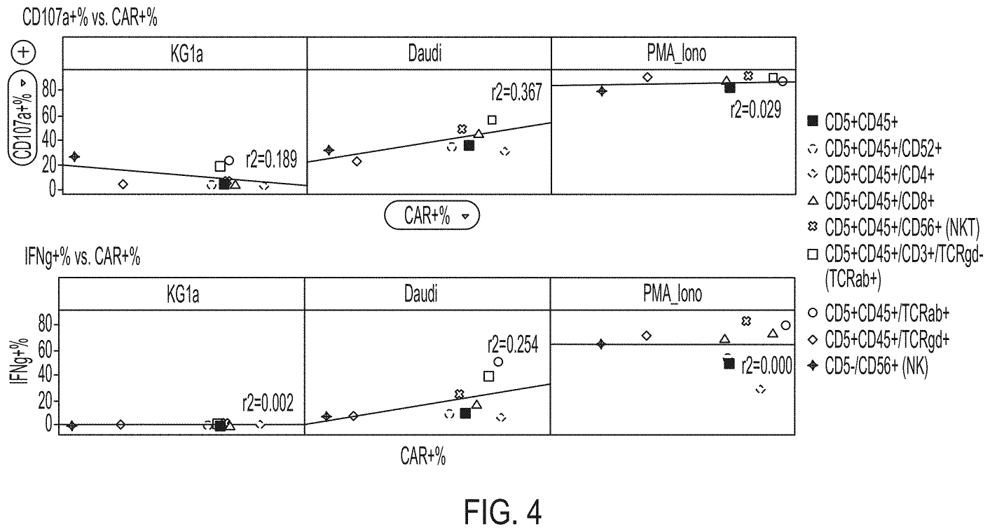

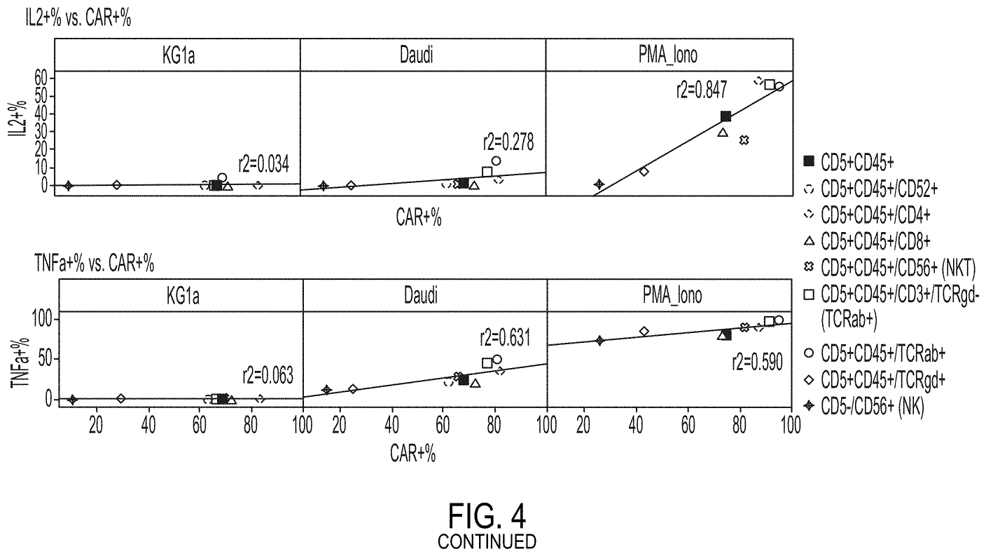

CD19 CAR T cells were co-cultured with CD19+ or CD19− target cells for 6 hrs as described above. The results in FIG. 4 show that for target-specific stimulation by CD19+ Daudi cells, CAR expression levels as determined by intracellular staining show highest correlation with TNFα (Pearson r=0.79, regression r\hat{ }2=0.631, p<0.02) but weak to no correlation with any other effector molecules tested (CD107a, IFNγ, IL2) across various immune cell subsets. When the correlations were examined in a T cell memory panel, the results exhibited less robust relationships (data not shown). Immune lineages with the highest levels of CAR CD19+ and CD107a were residual TCRαβ+ cells (comprising <0.5% of drug product after TRAC TALEN-mediated ablation) (80.6% CAR+, 64.8% CD107a+), CD8+ T cells (72.0% CAR+, 44.6% CD107a+), and NKT cells (65.4% CAR+, 48.6% CD107a+). We also observed that immune cell lineages with the lowest levels of CD107a induction despite high levels of CAR expression was CD4+ T cells (81.7% CAR+, 30.4% CD107a+). Notably, the CD107a was not induced when CAR T cells were co-cultured with off-target tumor cells, especially in the TCRαβ+ lineages, which indicates that this residual population is not exerting an off-target allogeneic graft versus host effect (GvH). For pan-CAR T cell specific stimulation with PMA and Ionomycin, IL-2 was significantly correlated with CAR expression (r=0.92, P<0.0005), followed by TNFα mostly driven by the CD4+ population (r=0.77, P<0.02). Additionally, this same trend specifically within IL-2 was observed across T cell memory populations (data not shown).

In summary, CAR expression levels (% CAR+) significantly correlated with TNFα expression levels but not IFNγ or IL2 across the immune cell lineages, and CD107a induction was highest in TCRαβ+, CD8+ and NKT cells. These results underscore the relevance of CD107a as being a highly correlative proxy of cytokine-induced allogeneic CAR T drug product across the different immune cell lineages responding to target cells. It also obviates the need to perform intracellular cytokine staining which would be prohibitive to perform in the GMP setting. In addition, the discovery that CD107a can be used as a highly correlative proxy also improves upon the current methods of directly measuring individual cytokine by ELISA (enzyme linked immunosorbent assay). Such direct measurement would not specify which cellular subsets in the drug product are capable of secreting cytokines.

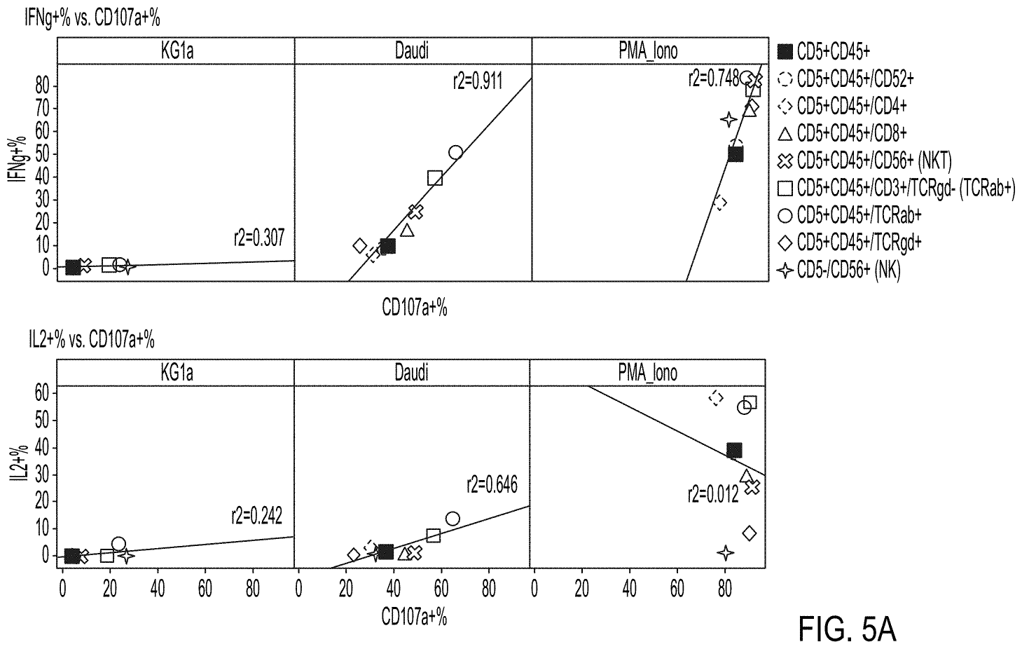

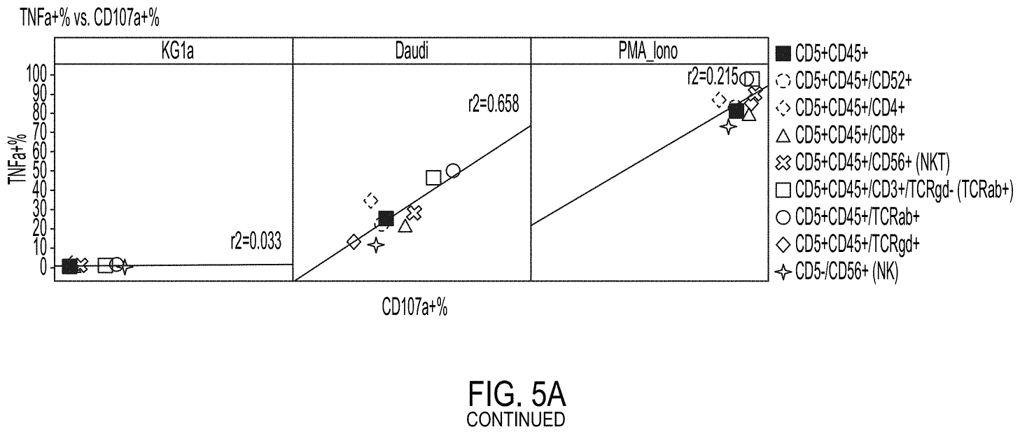

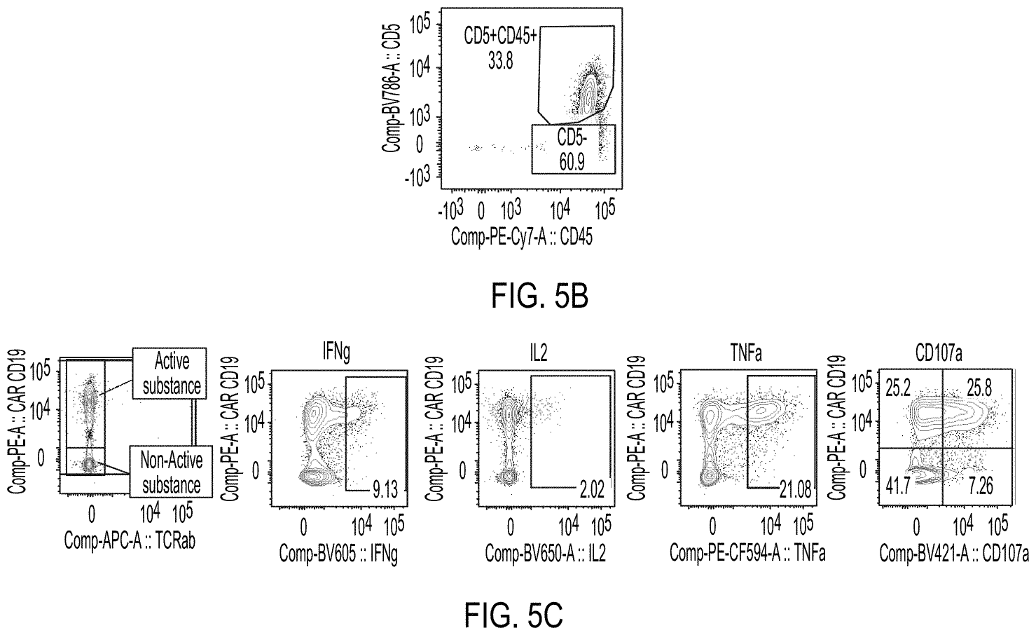

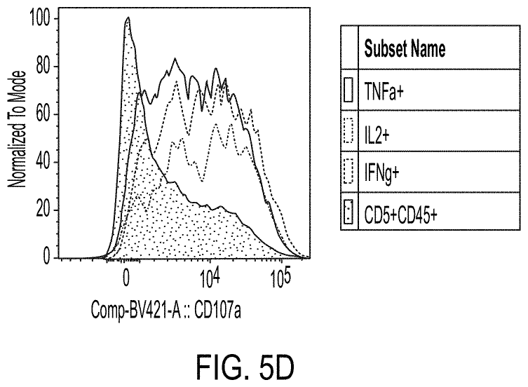

Although we observed no significant correlation between CAR positivity and CD107a intracellular staining in Daudi-cocultured CAR T cells, CD107a expression levels showed robust correlation with all three cytokines: IFNγ (r=0.91, p<0.0001), TNFα (r=0.66, p<0.01), and IL2 (Pearson r=0.8, r\hat{ }2=0.646, p<0.01), in CAR T cells cocultured with Daudi cells, across various immune cell subsets. See FIG. 5A and FIG. 5D. As shown in FIG. 5C, in all CAR T cells stimulated with tumor cells, most of the positive CD107a staining coincided with positive CAR staining (see the rightmost panel under FIG. 5C). The immune lineage with the highest induction of CD107a were residual TCRαβ+ cells (comprising <0.5% of drug product after TRAC TALEN®-mediated ablation) (64.8% CD107a+, 50.9% IFNγ+, 50% TNFα+, 13.9% 1L2+). Additional immune lineages with the highest induction of CD107a were NKT cells (48.6% CD107a+, 24.8% IFNγ+, 28.3% TNFα+, 1.07% 1L2+), and CD8+ T cells, (44.6% CD107a+, 16.7% IFNγ+, 21.5% TNFα+, 0.69% 1L2+). Immune cell lineages with the lowest levels of CD107a induction were TCRγδ T cells (23.2% CD107a+), which also demonstrated the least amount of CAR+ staining (24.9% CAR+) and cytokine positive events (7.46% IFNγ, 12.9% TNFα+, 0.23% IL2). NK cells (CD5−CD56+) cells also showed similarly low levels of CD107a induction and CAR expression (32.45% CD107a+, 14.41% CAR+). For pan-CAR T cell specific stimulation with PMA and Ionomycin, CD107a was less correlated with any of the cytokines across all lineage subsets as compared to the robust correlations observed with Daudi CD19+ specific stimulation. The results indicate that CD107a is specifically induced upon antigen-specific recognition.

In summary, the results show that CD107a strongly correlated with the extent of all three cytokine inductions (IFNγ, TNFα, and IL2) far more than the extent of CD19 CAR expression, which may be attributed to different CAR+% in different lineages of cells as shown in FIG. 4.

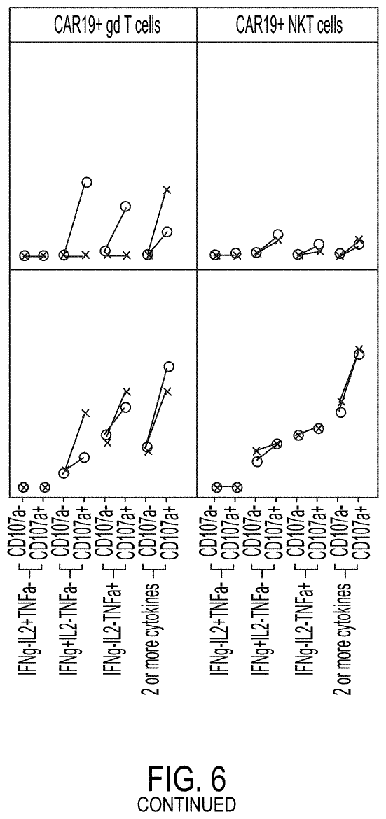

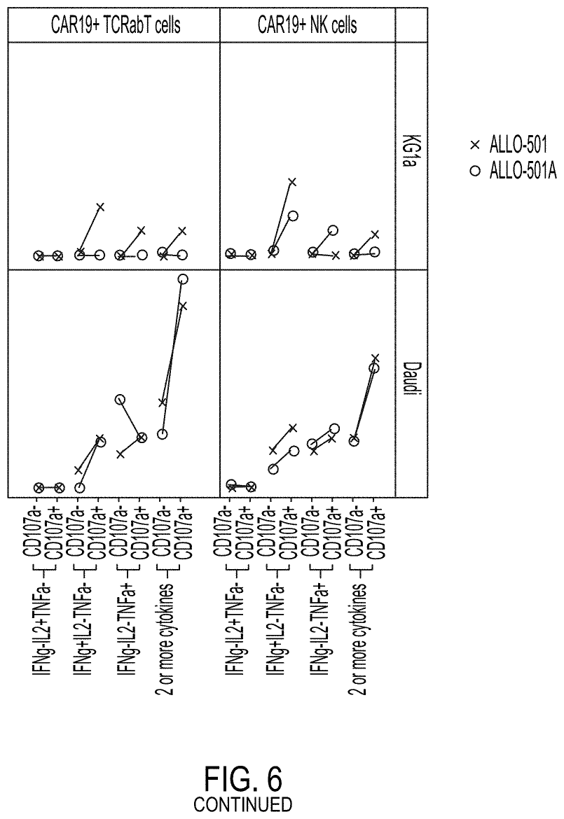

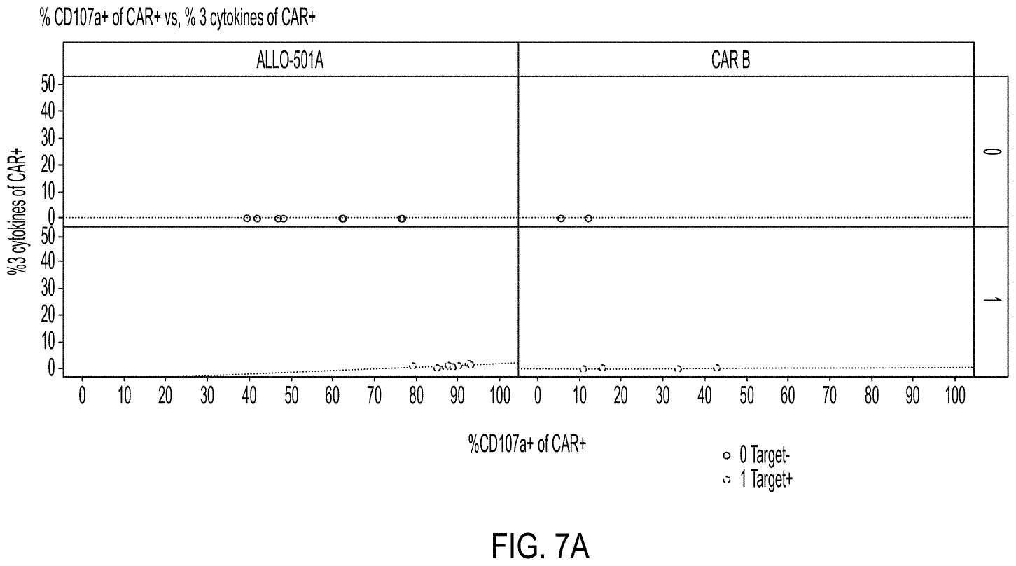

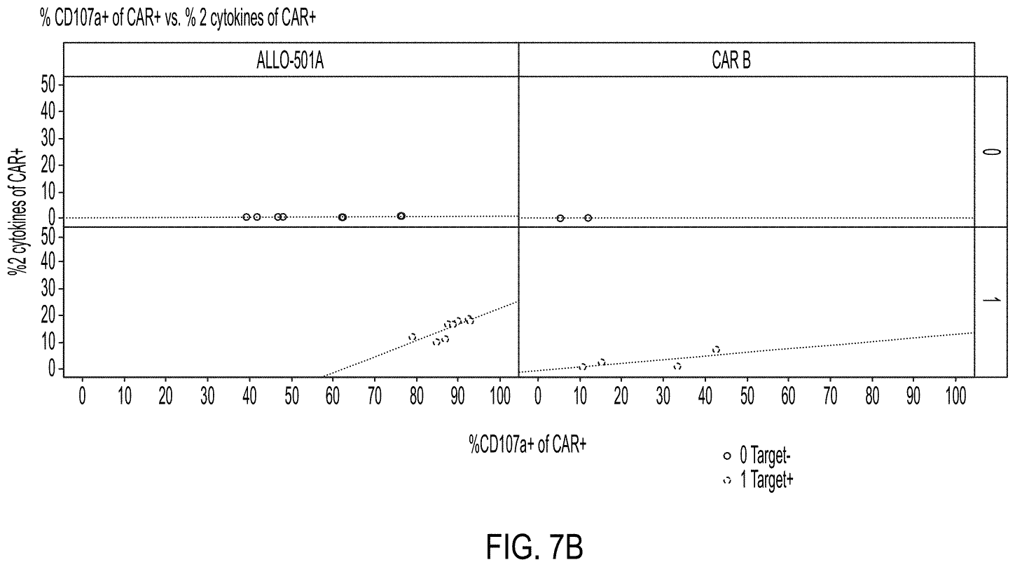

The correlations between surface CD107a and multiple effector cytokines, i.e., polyfunctionality, were evaluated by simple linear regression in CD19 CAR T cells (ALLO-501A) and in CAR T cells that are specific for a non-CD19 target B (CAR B). After co-culturing with cells that express the respective target (Daudi, ACHN-GFP or Raji cells) verses target negative control cells for 6 hours, correlations between surface expression of CD107a and polyfunctionality of CAR T cells were observed. Polyfunctionality was demonstrated by intracellular staining of two out of three or three out of three cytokines TNFα, IL2 and INFy in CAR T cells. The strength of correlation from the simple linear regression analysis is determined as follows: r=0.00-0.19, no/very weak correlation; r=0.20-0.39, weak correlation; r=0.40-0.59, moderate correlation; r=0.60-0.79, strong correlation; and r=0.80-1.00, very strong correlation. As shown in FIGS. 7A-7B, strong correlations were observed for CD19 CAR T cells when three or two cytokines were examined (r=0.6223 for three cytokines and r=0.7740 for two cytokines) and the correlation is statistically significant for the two-cytokine data (p=0.0242*). A strong correlation was also observed in CAR B T cells when two cytokines were examined (r=0.6785) though the data are not statistically significant. A weak correlation was observed for CAR B T cells when three cytokines were examined (r=0.3761). No to weak correlations were observed when the cells were co-cultured for 18 hrs (data not shown).

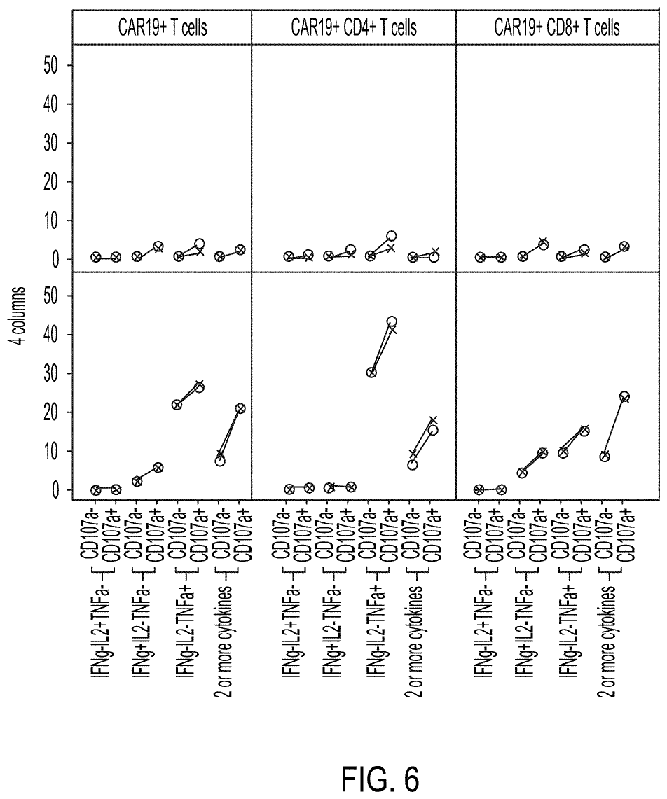

We next examined within each CD19 CAR+ immune cell lineage, the ability of Daudi-cocultured CD107a+ cells or CD107− cells to express each individual or two or more of the three cytokines, IFNγ, IL2 and TNFα. As shown in FIG. 6, cells expressing just IFNγ (i.e., IFNg+IL2−TNFα−) were specifically enriched in CD107a+ as compared to CD107a− populations, across nearly all CAR+ lineages measured—total events, T cells, CD8+ T cells, TCRγδ T cells, NKT cells, TCRαβ+, and NK cells. The exception was CAR19+CD4+ T cells. See FIG. 6. Cells expressing just TNFα (i.e., IFNγ-IL2-TNFα+) also showed the specific enrichment in CD107a+ versus CD107a− populations across nearly all CAR19+ lineages measured—total events, T cells, CD8+ T cells, TCRγδ T cells, NKT cells, TCRαβ+, and NK cells. The exception was residual TCRαβ+ T cells. Most strikingly, cells expressing two or more combinations of IFNγ, IL2, and TNFα were significantly enriched in CD107a cells across all CAR+ subsets.

In summary, CD107a expression demarcates target-specific induction of two or more combinations of IFNγ, IL2, and TNFα across all CAR+ immune subsets measured and CD107a surface expression can be used as an indicator for polyfunctionality of allogeneic CAR T cells. See FIG. 3 Panel I, analyzed from the “AS” population of Panel E (“Active Substance” as defined for regulatory purposes as viable CAR+/TCRαβ− T cells). Due to the limited sample size, statistics were not performed for this study, although the CAR T cells derived from matching donor cells harboring either Allo-501 or Allo-501A showed similar patterns of cytokine induction across CD107a populations. Thus, a correlation exists between CD107a+ and effector cytokine induction in CAR+ cells. This observation further established that surface CD107a can be used as an indicator for multifunctionality of CAR T cells.

To assess the unbiased clustering of effector molecules according to the expression distribution of lineage markers, t-distributed stochastic neighbor embedding (t-SNE) analysis and FlowSOM, a self-organizing map clustering algorithm, were performed on the down-sampled viable, singlet, CD5+, CD45+ populations. We found that using unsupervised clustering approaches, CD107a specifically demarcates a cluster of cells that are polyfunctional with combinations of cytokines expressed (IFNγ+, 1L2+, and TNFα+) (data not shown).

In conclusion, the results presented show that degranulation marker CD107 can serve as indicator for target antigen-specific induction of cytokines and thus a proxy for the assessment of potency, of allogeneic CAR T cells. The surface expression of CD107 upon degranulation is a more accessible biomarker, and the detection of which bypasses the cumbersome steps of direct measurement of intracellular staining of each induced cytokine that are often difficult to implement in large scale manufacturing settings.

1. A method of analyzing a population of immune cells, wherein the population of immune cells has been engineered to introduce one or more genetic modifications at the TCRα, and/or TCRβ locus to reduce or impair TCRαβ surface expression, the method comprising the steps of:
a) Obtaining or measuring viable CD45+ cells from the population of immune cells;
b) Obtaining or measuring CD5+/CD3+ cells from the cells in step a); and
c) Measuring or determining a percentage or amount of CD3+/TCRγδ− cells from the cells in step b),
wherein the percentage or amount of CD3+/TCRγδ− cells in step c) indicates a percentage or amount of TCRαβ+ T cells present in the population of immune cells.

2. A method of measuring a percentage or amount of TCRαβ+ T cells in a population of immune cells, wherein the population of immune cells has been engineered to introduce one or more genetic modifications at the TCRα, and/or TCRβ locus to reduce or impair TCRαβ surface expression, the method comprising the steps of:
a) Obtaining or measuring viable CD45+ cells from the population of immune cells;
b) Obtaining or measuring CD5+/CD3+ cells from the cells in step a); and
c) Measuring or determining a percentage or amount of CD3+/TCRγδ− cells from the cells in step b),
wherein the percentage or amount of CD3+/TCRγδ− cells in step c) indicates the percentage or amount of TCRαβ+ T cells in the population of immune cells.

3. The method of claim 1, wherein the population of immune cells has been engineered to express a chimeric antigen receptor (CAR).

4. The method of claim 1, wherein the population of immune cells is peripheral blood mononuclear cells (PBMC) or a population of CD4+ and/or CD8+ T cells.

5. The method of claim 1, wherein the percentage or amount of TCRαβ+ T cells is determined by subtracting the percentage or amount of CD3+/TCRγδ+ cells from the population of CD5+/CD3+ cells in step b).

6. A method of analyzing a population of immune cells that has been engineered to express a CAR, comprising the step of measuring surface CD107 of the CAR T cells after antigen stimulation, wherein an increased level of surface CD107 as compared to a level before antigen stimulation indicates polyfunctional CAR T cells.

7. The method of claim 6, wherein the increased level of surface CD107 is an increased percentage or an increased mean/medium fluorescence intensities of surface CD107.

8. The method of claim 6, wherein the polyfunctional CAR T cells secret higher level of TNFα after antigen stimulation as compared to CAR T cells that are not polyfunctional.

9. The method of claim 6, wherein the CAR T cells have been engineered to introduce one or more genetic modifications at the TCRα and/or TCRβ locus to reduce or impair TCRαβ surface expression.

10. The method of claim 6, further comprising measuring one or more cytokines selected from the group consisting of INFγ, TNFα, IL2, GM-CSF, CXCL1, IL-1b, IL-2, IL-4, IL-5, IL-6, IL-7, IL-8, IL-9, IL-10, IL-12, IL-17, IL-21, IL-22, IL-23, CXCL11, Mip1a, Mip1b, Mip3a, TNFb, Perforin, Granzyme A, Granzyme B, Granzyme H, CCL11, IP-10, CCL5, TGFb, sCD137, sCD40L, MCP-1, and MCP-4.

11. The method of claim 6, wherein the CAR T cells is stimulated by co-culturing the CAR T cells with target cells expressing an antigen of the CAR.

12. The method of claim 11 wherein the target cells are tumor cells.

13. The method of claim 6, wherein the level of surface CD107 is measured by flow cytometry.

14. The method of claim 6, wherein CD107 is CD107a and/or CD107b.

15. The method of claim 6, wherein CD107 is measured about 4 hours, about 5 hours, about 6 hours, about 7 hours or about 8 hours after antigen activation.

16. A method of analyzing a population of immune cells, wherein the population of immune cells has been engineered to introduce one or more genetic modifications at the TCRα, and/or TCRβ locus to reduce or impair TCRαβ surface expression, and wherein the population of lymphocytes has been engineered to express a chimeric antigen receptor (CAR), the method comprising the steps of:
a) measuring or determining a percentage or amount of TCRαβ+ T cells in the population of immune cells according to the method of claim 1; and
b) measuring in the population of immune cells:
i) a percentage or amount of CAR+ T cells; and/or
j) level of surface CD107 after antigen stimulation.

17. The method of claim 16, wherein an increased level of surface CD107 after antigen stimulation as compared to a level before antigen stimulation indicates polyfunctional CAR T cells.

18. The method of claim 17, wherein the increased level of surface CD107 is an increased percentage or an increased mean/medium fluorescence intensities of surface CD107.

19. The method of claim 16, wherein CD107 is CD107a and/or CD107b.

20. The method of claim 16, wherein the percentage or amount of CAR+ T cells is measured using an anti-id antibody.

21. The method of claim 16, wherein CD107 is measured about 4 hours, about 5 hours, about 6 hours, about 7 hours or about 8 hours after antigen activation.

22. The method of claim 16, wherein the population of immune cells is PBMC or a population of CD4+ and/or CD8+ T cells.

23. The method of claim 16, wherein the percentage or amount is measured by flow cytometry.

24. The method of claim 16, wherein the population of immune cells are obtained from a healthy donor.

25. The method of claim 16, wherein the CAR T cells are allogeneic CAR T cells.

26. The method of claim 1, wherein the immune cells express a CD19 CAR.

27. The method of claim 17, further comprising a step of filling the population of immune cells into one or more containers, if the amount or percentage of TCRαβ+ T cells is no more than a predetermined threshold and/or if the population of immune cells comprises polyfunctional CAR T cells.

28. A method of preparing a drug product comprising engineered immune cells, said method comprising the method of claim 1.

29. A kit or an article of manufacture for analyzing a CAR T cell comprising one or more reagents for detecting CD3 and TCRγδ.

30. The kit or article of manufacture of claim 29, further comprising one or more reagents for detecting CD45, CD5, CD52, CD107a and/or a CAR.

31. The kit or article of manufacture of claim 29, wherein the one or more reagents comprise an antibody.

32. The kit or article of manufacture of claim 29, wherein the one or more reagents are conjugated with a detectable label.

33. The kit or article of manufacture of claim 32, wherein the detectable label is selected from the group consisting of a fluorescent label, a photochromic compound, a proteinaceous fluorescent label, a magnetic label, a radiolabel, and a hapten.

34. The kit or article of manufacture of claim 33 wherein the fluorescent label is selected from the group consisting of an Atto dye, an Alexafluor dye, quantum dots, Hydroxycoumarin, Aminocouramin, Methoxycourmarin, Cascade Blue, Pacific Blue, Pacific Orange, Lucifer Yellow, NBD, R-Phycoerythrin (PE), PE-Cy5 conjugates, PE-Cy7 conjugates, Red 613, PerCP, TruRed, FluorX, Fluorescein, BODIPY-FL, Cy2, Cy3, Cy3B, Cy3.5, Cy5, Cy5.5, Cy7, TRITC, X-Rhodamine, Lissamine Rhocamine B, Texas Red, Allophycocyanin (APC), APC-Cy7 conjugates, Indo-1, Fluo-3, Fluo-4, DCFH, DHR, SNARF, GFP (Y66H mutation), GFP (Y66F mutation), EBFP, EBFP2, Azurite, GFPuv, T-Sapphire, Cerulean, mCFP, mTurquoise2, ECFP, CyPet, GFP (Y66W mutation), mKeima-Red, TagCFP, AmCyan1, mTFP1, GFP (S65A mutation), Midorishi Cyan, Wild Type GFP, GFP (S65C mutation), TurboGFP, TagGFP, GFP (S65L mutation), Emerald, GFP (S65T mutation), EGFP, Azami Green, ZsGreenl, TagYFP, EYFP, Topaz, Venus, mCitrine, YPet, TurboYFP, ZsYellow1, Kusabira Orange, mOrange, Allophycocyanin (APC), mKO, TurboRFP, tdTomato, TagRFP, DsRed monomer, DsRed2 (“RFP”), mStrawberry, TurboFP602, AsRed2, mRFP1, J-Red, R-phycoerythrin (RPE), B-phycoeryhring (BPE), mCherry, HcRed1, Katusha, P3, Peridinin Chlorophyll (PerCP), mKate (TagFP635), TurboFP635, mPlum, and mRaspberry.

35. The kit or article of manufacture of claim 29, wherein the one or more reagents are used for flow cytometry.


SEQUENCE LISTING (2 sequences)

SEQ ID NO: 1 - protein, 475 aa, Artificial Sequence
  Description of Artificial Sequence Synthetic polypeptide
EVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 495 aa, Artificial Sequence
  Description of Artificial Sequence Synthetic polypeptide
METDTLLLWVLLLWVPGSTGEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR